(19) United States
(12) Patent Application Publication
(10) Pub. No.: US 2026/0000884 A1
(43) Pub. Date: Jan. 1, 2026

(54) PERCUTANEOUSLY APPLIED BLOOD PUMP CAPABLE OF CHRONIC SUPPORT

(71) Applicant: inQB8 Medical Technologies, LLC, Winchester, MA (US)

(72) Inventors: Arshad Quadri, West Hartford, CT (US); J. Brent Ratz, Winchester, MA (US); Alexander H. Cooper, Costa Mesa, CA (US)

(21) Appl. No.: 18/869,628

(22) PCT Filed: May 24, 2023
(86) PCT No.: PCT/US2023/023431

§ 371 Date: Nov. 26, 2024

Related U.S. Application Data
(60) Provisional application No. 63/365,470, filed on May 27, 2022.

(51) Int. Cl. A61M 60/139; A61M 60/237; A61M 60/416; A61M 60/808; A61M 60/81; A61M 60/865; A61M 60/875; A61M 60/876

(52) U.S. Cl. CPC A61M 60/139; A61M 60/237; A61M 60/416; A61M 60/808; A61M 60/81; A61M 60/865; A61M 60/875; A61M 60/876; A61M 2205/0294; A61M 2205/3334; A61M 2205/583

(57) ABSTRACT

A system and method for increasing cardiac output of a heart patient and/or diuresis is disclosed. The system can include a fluid pump with an expandable housing and an impeller. The impeller can be non-obstructive to at least some blood flowing between impeller blades of the impeller. The impeller can be housed in an expandable housing that is open to incoming flow which also reduces obstruction to blood flow. The fluid pump can be placed within and powered to rotate an impeller of the fluid pump in a first direction. The fluid pump can be switched to an unpowered state. In the unpowered state the impeller can be rotated by blood flowing through the fluid pump to reduce the obstructive effect of the impeller and/or to engage a power generating and/or physiologic conditions sensing mode.

### INCORPORATION BY REFERENCE TO ANY PRIORITY APPLICATIONS

The present application is a National Phase Application of PCT/US2023/023431 filed May 24, 2023, titled PERCUTANEOUSLY APPLIED BLOOD PUMP CAPABLE OF CHRONIC SUPPORT, which claims priority to U.S. Provisional Patent Application No. 63/365,470, filed May 27, 2022, which is incorporated herein by reference. Any and all applications for which a foreign or domestic priority claim is identified in the Application Data Sheet as filed with the present application are hereby incorporated by reference under 37 CFR 1.57.

### BACKGROUND

### Field

This application is directed to a heart function support system configured to be applied percutaneously and configured to provide chronic support in some applications.

### Description

Congestive heart failure (CHF) is a complex, debilitating condition that involves several interrelated organs and systems. One symptom in heart failure patients is excess fluid in the circulatory system, or overload. Excess fluid can produce high blood pressure, against which the heart pumps to supply blood to the lungs and other organs. Excessive arterial blood pressure in a CHF patient (i.e., afterload) requires the heart to work much harder than in a healthy person. In the long run, this can lead to the heart becoming enlarged which can, in a severe case, prevent leaflets of heart valves of from coapting, among other complication. Excess fluid load can lead to high venous blood pressure, e.g., high central venous pressure (CVP). Excess CVP corresponds to high pre-load which can burden heart function leading to progression of CHF.

In a healthy person the kidneys regulate blood fluid levels. Excess fluid is eliminated in urine produced by the kidneys. Accordingly, one approach to treating a patient with fluid overload is the induce diuresis. Diuresis is typically induced using pharmaceutical agents, called diuretics. The use of diuretics can increase urination, bringing down fluid load. Diuretics have shortcomings such as a reliance on patient compliance, which can be inconsistent. Also, diuretics are typically systemically administered, resulting in various side effects. Patients can become less responsive to diuretics over time as body systems adapt to the presence of the agent.

Catheter based blood pumps have been proposed for assisting the heart in circulation. Initial approved indications for catheter pumps include in connection with cardiogenic shock and high risk percutaneous coronary interventions. Some catheter pumps have been described that would reside in the vasculature. In some cases, blood pumps have been described with pumping elements disposed in the vasculature while inlets are disposed in the heart.

### SUMMARY

There is a need for heart function support systems configured to be applied percutaneously that can provide more effective treatment of heart failure patients.

In some embodiments of the disclosure, a method for increasing cardiac output of a heart patient and/or diuresis is disclosed that comprises placing a fluid pump within a blood vessel of the patient. The fluid pump can be powered to rotate an impeller of the fluid pump in a first direction. The fluid pump can be switched to an unpowered state. In the unpowered state the impeller can be rotated by blood flowing through the fluid pump. The impeller can be non-obstructive to at least some blood flowing between impeller blades of the impeller. The impeller can be housed in an expandable housing that is open to incoming flow which also reduces obstruction to blood flow.

In some embodiments, a system for chronic support of heart function is disclosed that comprises a motor sized for insertion into a blood vessel. The motor can comprise windings to generate magnetic fields when energized. The system can include a torque shaft assembly that can comprise a torque shaft and a rotor. The rotor can be rotated in response to the magnetic fields. The system can include an expandable housing that can include a first end and a second end opposite the first end. The first end can be coupled with the motor. A stent body can be disposed between the first and second end. The system can include an expandable propeller disposed in the expandable housing. The expandable propeller can comprise at least one propeller blade frame. The propeller blade frame can have a first end fixed to the torque shaft, and a second end opposite the first end. The second end can be slideable along the torque shaft. The impeller blade frame slideable end can be disposed adjacent to the motor. A fixed end of the impeller blade frame can be disposed on the torque shaft between the slideable end and the motor. In another arrangement, a fixed end can be disposed adjacent to a distal end of the torque shaft and a slideable end can be between the fixed end and the motor. The expandable impeller can include a tensile structure disposed along the at least one propeller blade frame. The tensile structure can extend radially inwardly from the at least one propeller blade frame. An angle of 90 degrees or less can be provided between a radial direction of the first end and a radial direction of the second end. When the windings of the motor are not generating magnetic fields, the expandable propeller can be configured to freely rotate in response to blood flow in the blood vessel.

In some embodiments, a system for enhancing cardiac output and/or diuresis through enhanced cardiorenal flow is disclosed that comprises a battery and a pump-generator unit. The pump-generator unit can include a housing that can include at least one wire coil assembly. The at least one wire coil assembly can be configured to convey current in response to a magnetic field and/or to generate a magnetic field in response to current conveyed therein. The pump-generator unit can include an expandable stent that can have a first end coupled with the housing, and a second end opposite the first end. A stent body can be disposed between the first end and the second end. The pump generator unit can include a shaft assembly that can include a shaft at least partially disposed in the expandable stent and a rotor rotatably coupled with the housing. The pump-generator unit can include at least one blade frame that can have a first end fixed to the shaft and a second end opposite the first end. The second end can be slideable along the shaft. There can be at least one tensile structure disposed along the at least one blade frame. The tensile structure can extend radially inwardly from the at least one blade frame when the at least one blade frame and the tensile structure are in an expanded state. In one operating state, blood flow onto the tensile structure can apply a load to the tensile structure resulting in a torque applied to the shaft and the rotor. The rotor can rotate in response to the torque causing a magnet in the rotor to generate a magnetic field to create a current in the at least one wire coil assembly. The current can be directed to the battery charge the battery.

In some embodiments, a system for chronic support of heart function is disclosed that comprises a pump assembly. The pump assembly can include an expandable stent and a motor coupled with a proximal end of the expandable stent or housing formed at least in part by the stent. A propeller can be disposed on a torque shaft disposed in the expandable housing. A power lead can be coupled with the motor at a distal end. The power lead has a proximal end disposed opposite the distal end, and a length between the proximal end and the distal end. The length can be sufficient to enable the pump assembly to be disposed in a blood vessel of a patient when the proximal end is disposed outside of a peripheral vessel in fluid communication with the blood vessel. The system can include a coil assembly that can include a support member enclosing an inductive coil. The inductive coil can be configured to couple with the proximal end of the power lead and the support member can be configured to be implanted minimally subcutaneously.

In some embodiments, the system for chronic support of heart function can include a motor sized for insertion into a patient, and a shaft assembly coupled with the motor. The shaft assembly can include a torque shaft. The system can include an expandable housing that can have a first end that can be disposed about at least a portion of the shaft assembly and a second end opposite the first end. The second end can be circumferentially self-supporting without axial or radial struts crossing the blood stream inward of the second end and thereby open to blood flow into or out of the expandable housing. A stent body can be disposed between the first and the second end. An expandable propeller can be disposed in the expandable housing. The expandable housing can include at least one propeller blade frame that can have a first end fixed to the torque shaft and a second end opposite the first end. The expandable propeller can include a tensile structure disposed along the at least one propeller blade frame. The tensile structure can extend radially inwardly therefrom when the expandable propeller is in an expanded state.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments.

FIG. 1 shows a schematic of the anatomy of a patient having a system for chronic support applied thereto;

FIG. 1A shows another schematic of the anatomy of a patient having a system for chronic support applied thereto;

FIG. 2 shows a schematic of blood vessels of a patient having a system for chronic support disposed therein;

FIG. 2A shows a schematic of the anatomy of a patient having an alternative system for chronic support applied thereto;

FIG. 2B shows a schematic of the anatomy of a patient having a second alternative system for chronic support applied thereto;

FIG. 3 shows a system for chronic support of the present disclosure;

FIG. 3A shows a schematic of a power assembly of a system for chronic support of the present disclosure;

FIG. 3B shows a schematic of an alternative power assembly of a system for chronic support of the present disclosure;

FIG. 4 shows an alternative embodiment of an impeller of a system for chronic support of the present disclosure having a helical impeller;

FIG. 4A shows shown an alternative embodiment of an impeller of a system for chronic support of the present disclosure having a screw shaped impeller;

FIG. 5 shows an alternative embodiment of a system for chronic support in a crimped state;

FIG. 6 shows the system of FIG. 5 in an expanded state;

FIGS. 7 and 8 show perspective and end views of an alternative embodiment of a system for chronic support in an expanded state;

FIGS. 9, 9A, and 10 show the expandable housing with the impeller removed to better illustrate techniques for providing the expandable housing;

FIGS. 11 and 12 show flat depictions of a first embodiment of a stent body of an expandable housing in a crimped state and in an expanded state;

FIG. 11A and 12A show flat depictions of a second embodiment of a stent body of an expandable housing in a crimped state and in an expanded state;

FIG. 13 shows an alternative impeller of a system for chronic support;

FIG. 13A shows a proximal end view of the impeller of FIG. 13;

FIG. 14 shows another alternative impeller for a system for chronic support, the impeller having a continuous body capable of wrapping about a central hub body thereof;

FIG. 15 shows another alternative system for chronic support of the present disclosure;

FIG. 15A is an end view of the system of FIG. 15;

FIG. 15B is a proximal perspective view of an alternative assembly for a system for chronic support of the present disclosure;

FIG. 15C shows a subassembly of the system of FIG. 15 with the expandable housing removed for clarity;

FIGS. 15D-15F show perspective, side, and end views of an impeller for the system of FIG. 15;

FIGS. 16A-16E show another embodiment of a cardiac support system and a method for deploying the system in the vasculature of a patient;

FIGS. 17 and 18 show another embodiment of a cardiac support system and a delivery system for deploying the cardiac support system in the vasculature of a patient;

FIGS. 19A-19B show another alternative impeller for a system for chronic support;

FIG. 20 shows another alternative embodiment of an impeller for a system for chronic support having a toroidal impeller; and

FIG. 21 shows a torque shaft of a system for chromic support of the present disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions may be provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

In the following detailed description, numerous specific details are set forth to provide a full understanding of the present disclosure. It will be apparent, however, to one ordinarily skilled in the art that embodiments of the present disclosure may be practiced without some of the specific details. In other instances, well-known structures and techniques have not been shown in detail so as not to obscure the disclosure.

Aspects of this disclosure are directed to systems and methods for increasing cardiac output of a heart patient or increasing diuresis. Certain aspects are directed to an open-ended configuration reducing the blood cell-device interactions in flow through the device. In other aspects a low angle impeller also can reduce the blood cell-device interactions in a non-operating state. Certain aspects are directed to percutaneously placing one or more non-obstructive freely rotating fluid pumps within a blood vessel of a patient. Other aspects are directed to supplying current to one or more non-obstructive freely rotating fluid pumps transcutaneously.

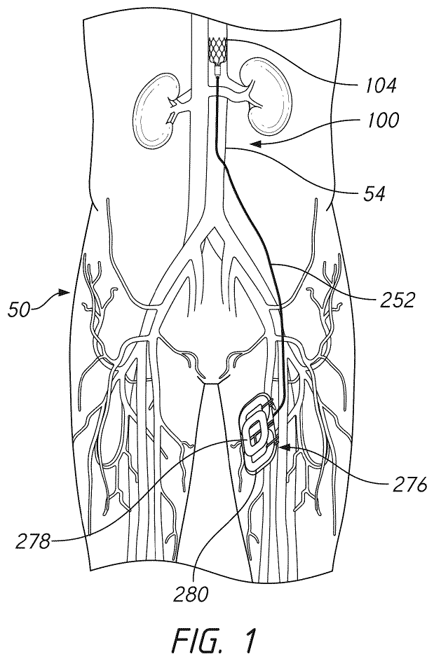

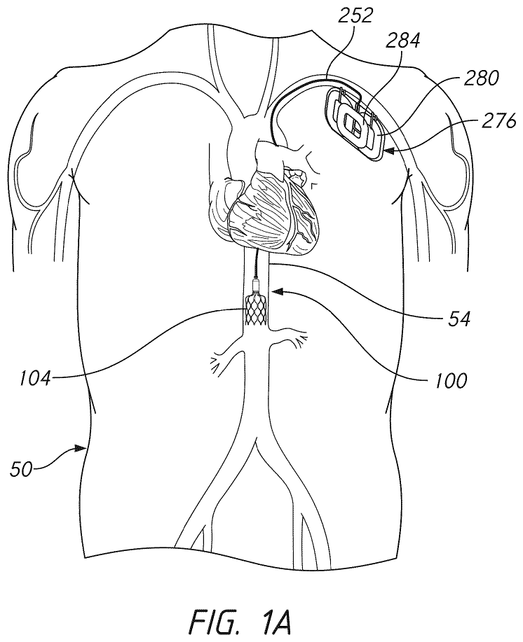

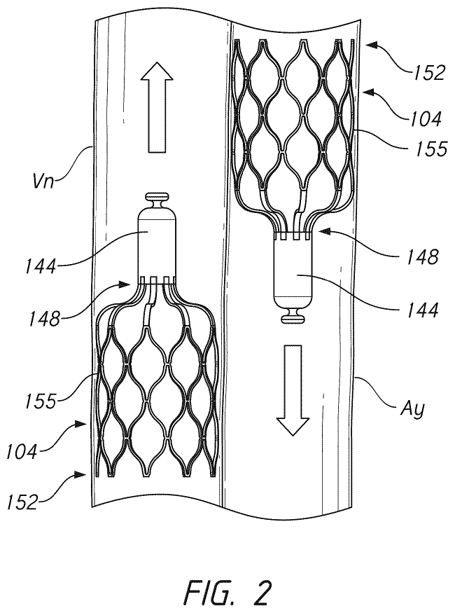

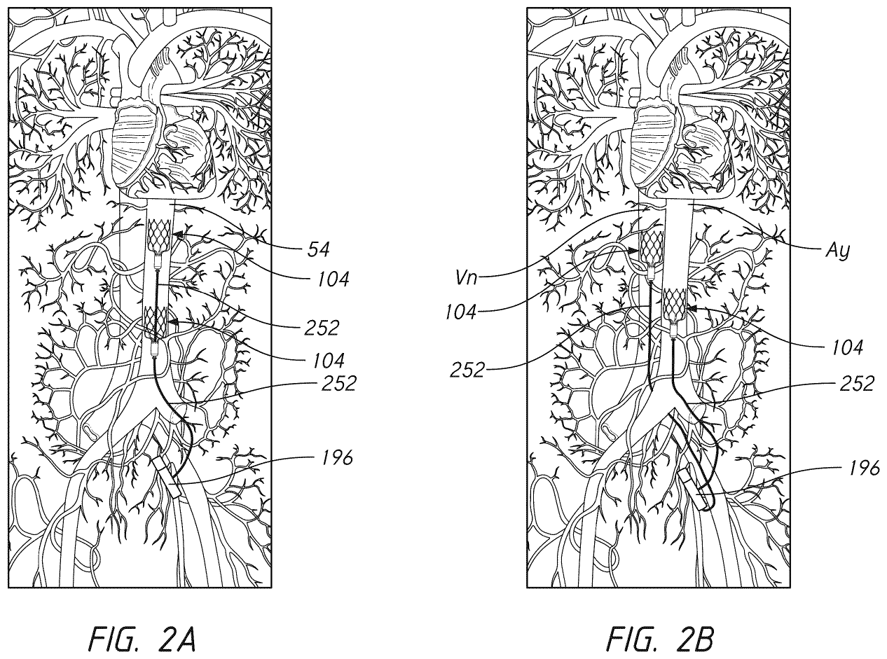

FIGS. 1 and 1A show a schematic of a patient 50 having a system for chronic support 100 applied thereto. As depicted, the system for chronic support 100 can be applied by placing a fluid pump 104 thereof in a major blood vessel 54, e.g., the descending aorta of the patient 50. The system for chronic support 100 can be applied through a peripheral blood vessel, such as a femoral artery as shown in FIG. 1, or the subclavian artery as shown in FIG. 1A. The system for chronic support 100 can be applied through other peripheral vessels of the leg or arm including subclavian artery, iliac artery, femoral vein, iliac vein, radial artery as non-limiting examples. The system for chronic support 100 can be fully implanted and can be powered by an implantable power supply device 276 as discussed further below. FIGS. 2, 2A, and 2B are directed to various additional systems and methods that can employ the fluid pump 104, as discussed further below. These figures are discussed below after a description of additional structural details of the system for chronic support 100.

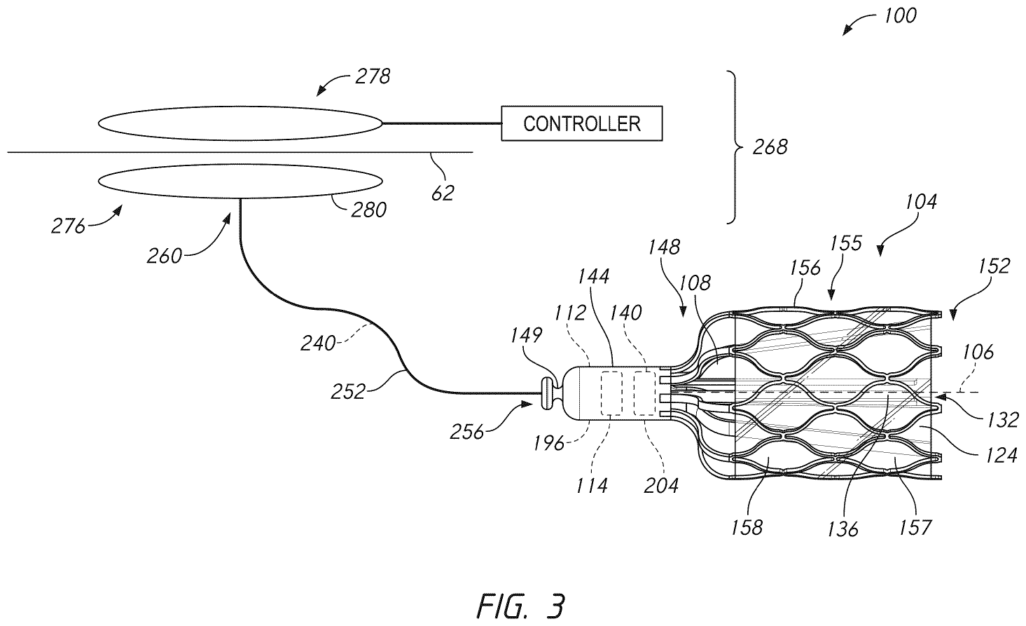

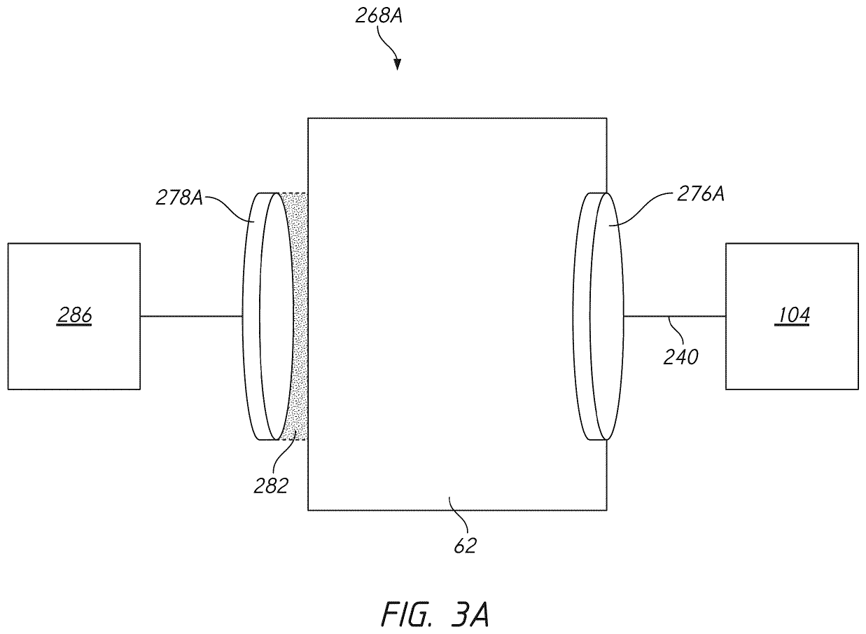

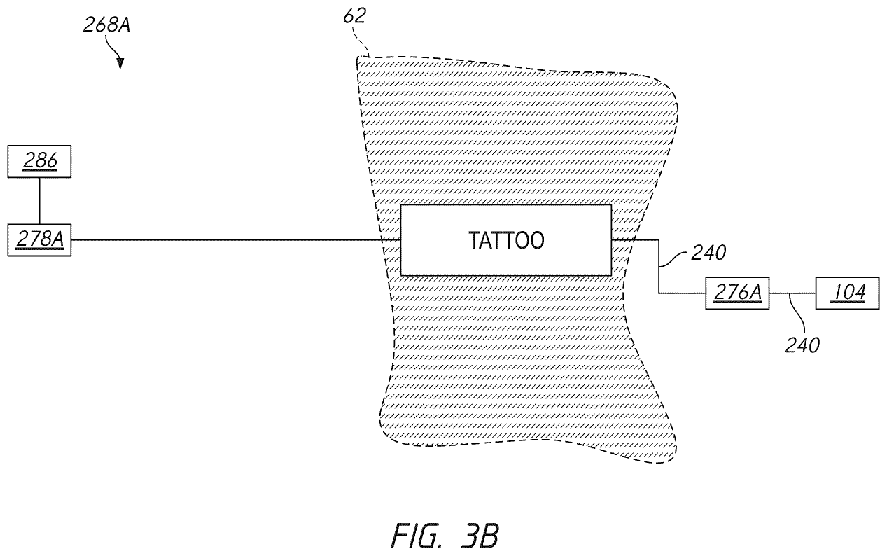

FIG. 3 shows that the system 100 includes a power lead 252 electrically connecting the power assembly 276 to the fluid pump 104. The power lead 252 can be configured to convey electrical current to the fluid pump 104 in some embodiments. In one variation, the fluid pump 104 can be mechanically actuated by a spinning cable in which case the power lead 252 is replaced with a sheath surrounding the cable. The sheath contains the spinning cable in a low friction environment and reduces the number of components disposed within the vasculature when applied because a motor for inducing torque can be disposed remote from an impeller of the fluid pump 104, e.g., subcutaneously but outside the vessels.

The fluid pump 104 includes an impeller 108 and a motor 112. The motor 112 can be disposed in a housing 144. The impeller 108 can be coupled with or mounted on a torque shaft assembly 132. The torque shaft assembly 132 can be coupled with the motor 112. The motor 112 can be rotatably coupled to the torque shaft assembly 132. The impeller 108 can be coupled to the torque shaft assembly 132 such that when the torque shaft assembly 132 rotates, the impeller 108 also rotates. The torque shaft assembly 132 can combined with the impeller 108 in a subassembly of the fluid pump 104. In some cases, the impeller 108 is disposed in an expandable housing 155. The expandable housing 155 can include a stent body 156. In some embodiments the expandable housing 155 includes a cover 157 that can enhance case of removal. In some applications the cover 157 is provided to reduce, slow, or even prevent endothelialization of the stent body 156 to enhance case of removal, as discussed further below. The cover 157 can contain some or all flow past the fluid pump 104 in a channel within the expandable housing 155. The cover 157 is omitted from a proximal portion of the expandable housing 155, e.g., leaving a portion of the stent body 156 uncovered in a proximal region through which blood can enter or exit the expandable housing 155.

The housing 144 can contain the motor 112 and a rotor 140. A first end 148 of the expandable housing 155, e.g., of the stent body 156, can be coupled the motor housing 144. The stent body 156 can extend to a second end 152 of the expandable housing 155 opposite the first end 148. The stent body 156 can be disposed between the first end 148 and the second end 152 of the expandable housing 155. The torque shaft assembly 132 and the impeller 108 can be disposed in the expandable housing 155, e.g., within the stent body 156. If provided, the cover 157 can be located outside of the stent body 156, e.g., around an outer surface of portion of the stent body 156. The stent body 156 can be disposed between the cover 157 and the impeller 108.

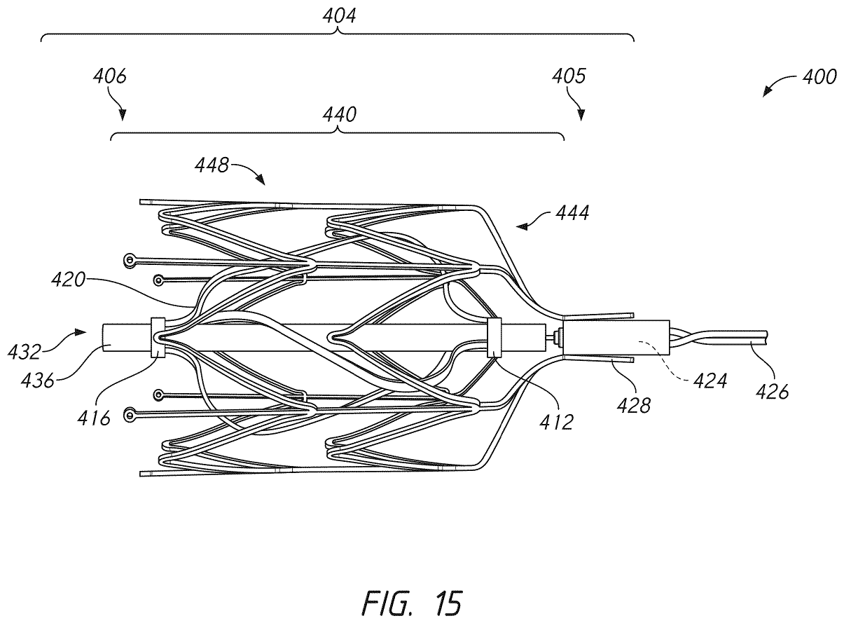

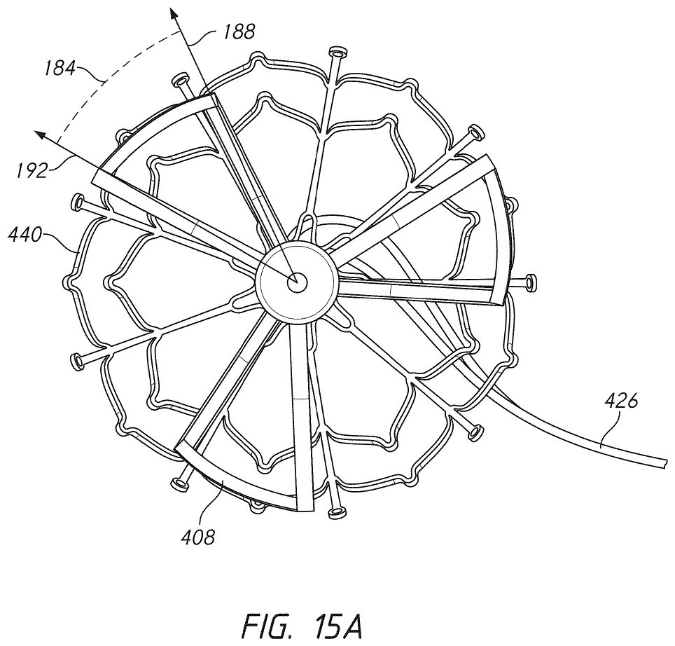

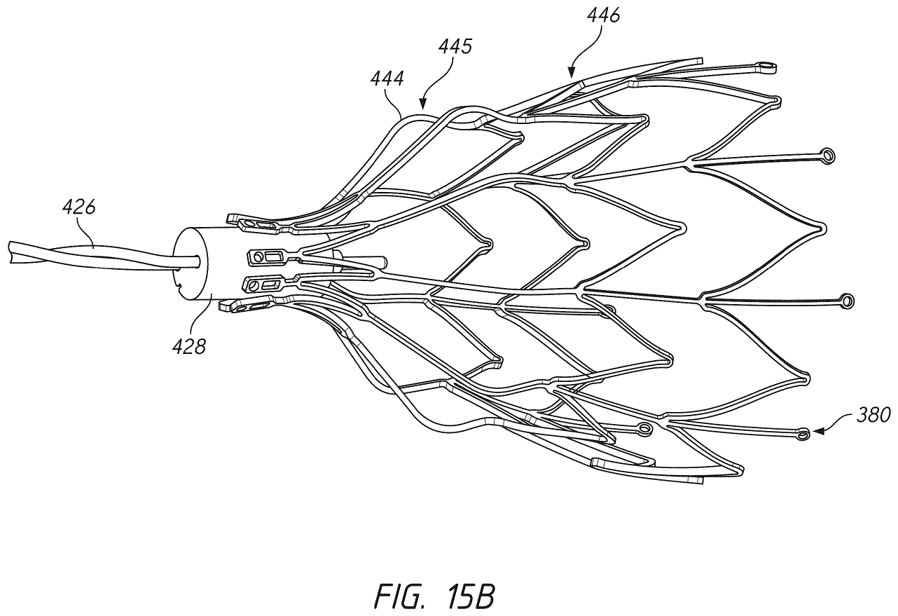

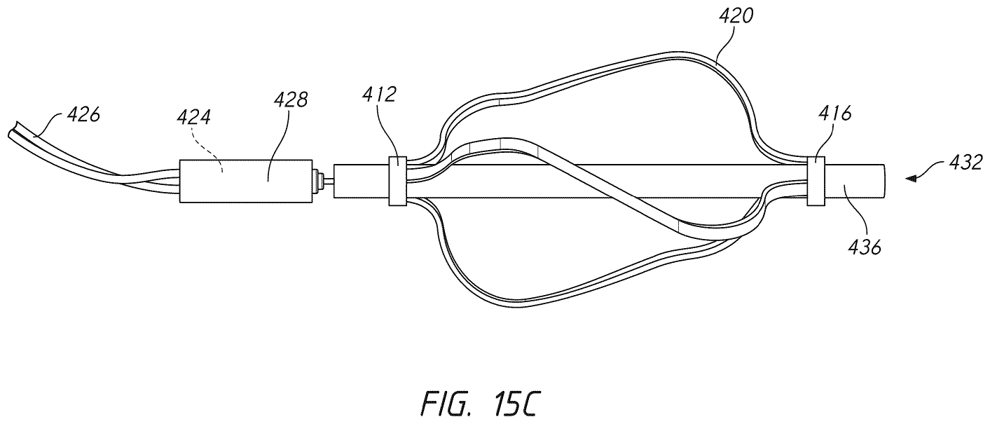

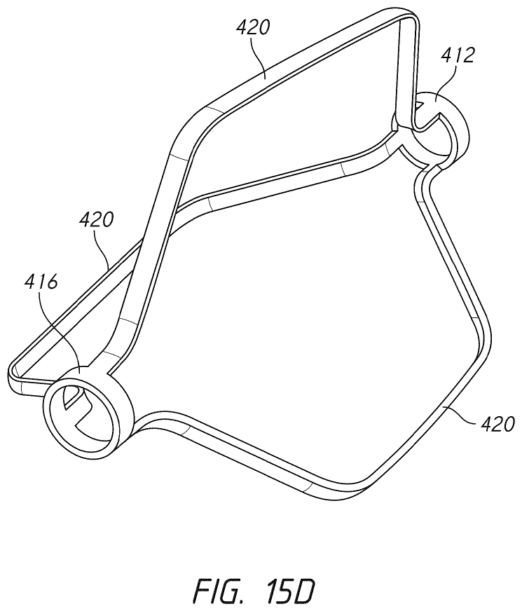

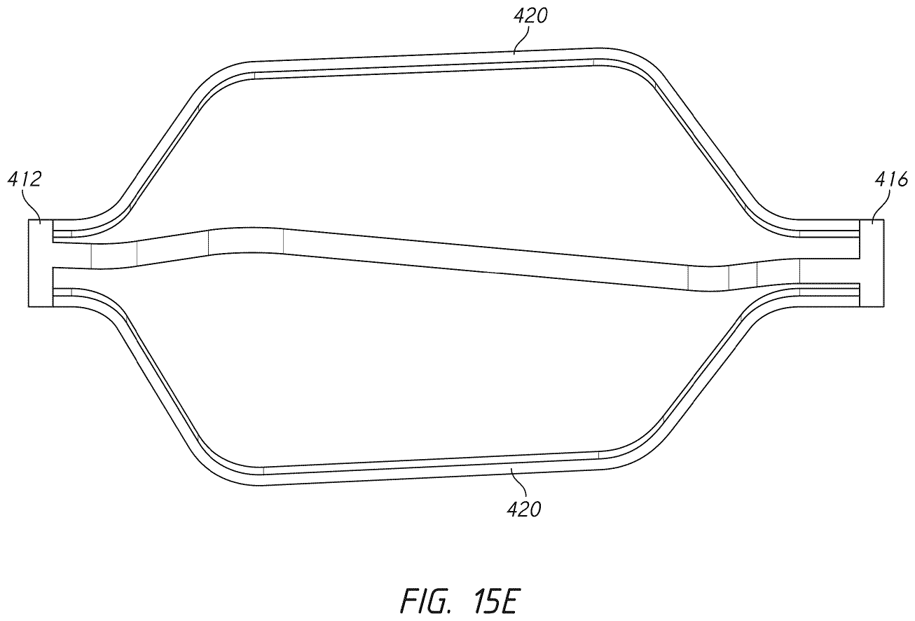

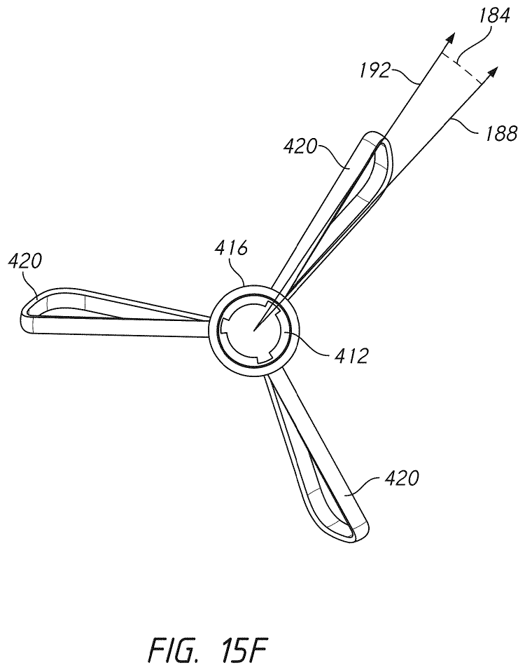

In some examples the expandable housing 155 is configured to provide an open space in which the impeller 108 can rotate. The expandable housing is open at the second end 152, for example. The stent body 156 can have a smaller diameter near the motor housing 144 and a larger diameter distal the smaller diameter portion. The stent body 156 can have a largest diameter at the second end 152 of the expandable housing 155. The stent body 156 can have an approximately cylindrical shape from the second end 152 proximally toward the motor housing 144. The stent body 156 can have a cylindrical shape from the second end 152 to a portion of the stent body 156 that tapers down to the profile, e.g., diameter, of the housing 144. In other embodiments, a more gradual tapered profile can be provided. As discussed further below in connection with FIG. 15B, modified embodiments of the stent body 156 can be provided wherein the stent body 156 generally tapers from the second end 152 towards the housing 144 of the motor 112.

FIG. 3 shows that the impeller 108 can have a tapered configuration in which the outermost point of the impeller 108 at each axial position is larger distal of an immediately adjacent portion proximal of the axial position. The impeller 108 is continuously wider in the radial direction from the proximal end to the distal end thereof. The largest radial dimension of the impeller 108 can be found at a distal end 120 of the impeller 108. In one configuration a tip gap between a radially outer-most portion of the impeller 108 and the inner periphery of the expandable housing 155 can vary along the length of the impeller 108 from a minimum gap at the distal end 120 of the impeller 108 to larger gaps proximal thereof. In this context, the tip gap is measured radially outward from the radially outermost part of the impeller 108 to the inner surface of the expandable housing 155, e.g., to the inner surface of the stent body 156. Preferably the minimum tip gap is on the order of about 5 percent to about 50 percent of a radius of the expandable housing 155, in some cases about 10 percent to about 40 percent, in some cases 25 percent to about 35 percent, in some cases about 30 percent of the radius of the expandable housing 155. In some embodiments, the stent body 156 and the impeller 108 can comprise one or more magnets with the same polarity. The magnets can be configured such that magnets on the stent body 156 repel magnets on the impeller 108, preventing or reducing the incidence or extent of contact between the impeller 108 and the stent body 156. In some embodiments, the magnets on the stent body 156 can be coupled with the motor 112, e.g., can induce magnetic fields that play a role in inducing rotation of the impeller 108. In one embodiment the entire rotational behavior of the impeller 108 when activated is induced by energizing coils coupled with or disposed around the expandable housing 155. In this and other embodiments disclosed herein unpowered rotation of the impeller 108 can be induced by the native blood flow. That rotation can be used to harvest energy as discussed further below.

Although a tip gap can be present in the usual spatial arrangement of the impeller 108 within the expandable housing 155, the cover 157 can encapsulate the stent body 156 at least in the region of the impeller 108 such that any occasional impingement of the impeller 108 on the expandable housing 155 does not involved the impeller 108 impacting a metallic structure (or other relatively hard portion) of the expandable housing 155. The cover 157 can have a lower hardness than the stent body 156. The cover 157 can be deformable (e.g., clastic) in some cases to reduce, delay or prevent wear of the impeller 108 within the expandable housing 155 in the event of occasional impact between the impeller 108 and the expandable housing 155.

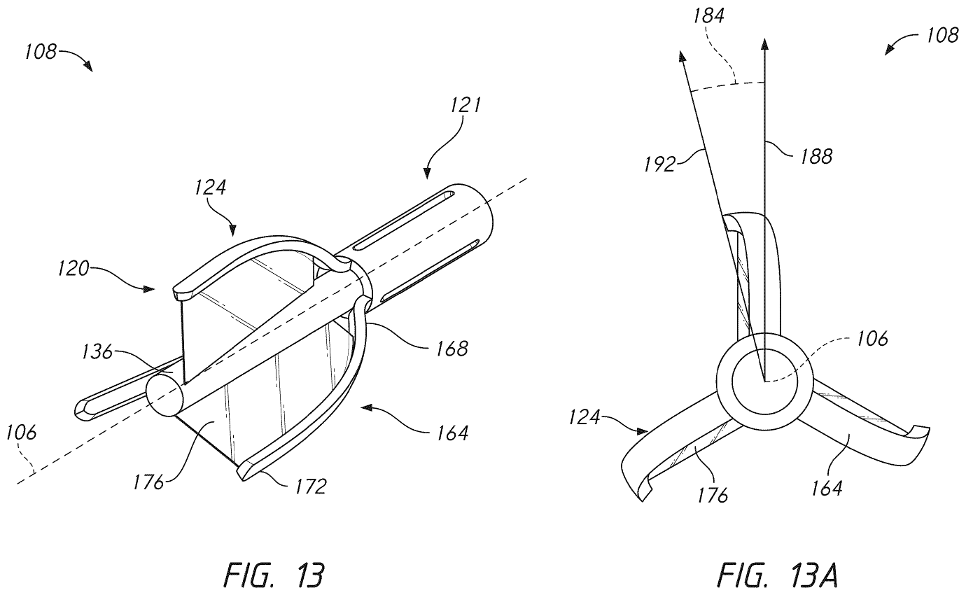

FIGS. 3 and 13-13A show additional aspects of embodiments of the impeller 108 and variations thereof. The impeller 108 can include at least one impeller blade 124. The at least one impeller blade 124 can include an impeller blade frame 164 and a tensile structure 176 coupled with the impeller blade frame 164. The impeller blade frame 164 can include a first end 168 and a second end 172. The first end 168 can be part of or coupled with a hub 121 disposed about the torque shaft 136. The structure of the impeller blade frame 164 of each blade can include a curved profile when viewed from the side of the impeller blade frame 164. For example, a relatively steep curvature can be provided at or adjacent to the first end 168. The impeller blade frame 164 of each blade can include a less-curved profile adjacent to the second end 172, e.g., a larger radius of curvature or even a straight segment adjacent to the second end 172. FIG. 13 shows that the impeller blade frame 164 can have a cantilever structure. The second end 172 of the impeller blade frame 164 can be self-supporting. The impeller blade frame 164 can be configured to self-expand to a free state. The free state or shape can be as seen in FIG. 13. The free shape of the impeller blade frame 164 alone can have a larger radial extent than that illustrated in FIG. 13 by virtue of a radially inward force applied by the tensile structure 176 when the tensile structure is stretched by the impeller blade frame 164. FIG. 13 shows that the impeller 108 can have three blades. In variations the impeller 108 can have more or fewer than three blades, e.g., 2 blades, four blades, fives blades, six blades, seven blades, or eight or more blades. In the illustrated embodiment, the impeller blades 124 are rigidly connected to the hub 121 so all of the blades rotate at the same time in the same direction. As discussed below, in one variation, contra-rotating impellers provide for some blades to rotate in a first direction while other blades rotate in a second direction opposite the first direction.

The tensile structure 176 can be coupled to the impeller blade frame 164 between the first end 168 and the second end 172 to form a majority of the blood propelling surface of the at least one impeller blade 124. The tensile structure 176 can be coupled to the impeller blade frame 164 and can extend from the first end 168 to the second end 172 of the impeller blade frame 164. The tensile structure 176 can be coupled at an end thereof opposite to the end coupled to the impeller blade frame 164. The tensile structure 176 can be coupled to the torque shaft assembly 132, e.g., to a torque shaft 136 of the torque shaft assembly 132 projecting distally of the hub 121.

The at least one impeller blade 124 can extend radially outward from a central axis 106 from the first end 148 of the housing to the second end 152 of the housing. An angle 184 can be provided between a radial direction of the first end 168 of the impeller 108 and radial direction of the second end 172 of the impeller blade 124 of the impeller 108. The tensile structure 176 can be coupled with the torque shaft assembly 132 at an angle to the central axis 106. For example, a distal connection portion of the tensile structure 176 can be coupled to the torque shaft 136 and can be offset by an angle relative to the position of connection of a proximal connection portion to the torque shaft 136. The offset angle can match the angle 184 in some embodiments. In some embodiments the tensile structure 176 does not extend entirely from the first end 168 to the second end 172 of the impeller blade frame 164 and the angle between the proximal and distal connection portions can be less than the angle 184, e.g., 5 percent, 10 percent 15 percent 20 percent or up to 25 percent less than the angle 184. The tensile structure 176 can be secured to the torque shaft assembly 132 with an adhesive. The tensile structure 176 can comprise a flange that can be inserted into a groove in the torque shaft assembly 132 to secure the tensile structure 176 to the torque shaft assembly 132. The tensile structure 176 can comprise a sleeve configured to wrap around the outside of the torque shaft assembly 132 to secure the tensile structure 176 to the torque shaft assembly 132. The tensile structure 176 can be formed of polyurethane (PU) or expanded polytetrafluoroethylene (ePTFE) or other suitable generally non-compliant polymeric material.

In some variations, the impeller 108 can include a first impeller and the fluid pump 104 can have a second impeller. The first impeller can be coupled with the motor 112 and the second impeller can be configured to rotate in a different manner than he first impeller 108. The second impeller can be couped with a second motor, for example. In these variations, the first impeller and the second impeller can be contra-rotating impellers, such that the first impeller and the second impeller rotate in opposite directions. For example, when the first impeller rotates clockwise (e.g., by the motor 112), the second impeller can rotate counterclockwise (e.g., by a second motor). The first impeller and the second impeller can be configured such that both the first impeller and the second impeller push fluid in the same direction when rotated by the motor 112. The angle 184 of the first impeller can be the opposite of the angle 184 of the second impeller. For example, the angle 184 of the first impeller can be 30 degrees, and the angle of the second impeller can be negative 30 degrees, where a positive angle is an angle in the counterclockwise direction, and a negative angle is in the clockwise direction.

The torque shaft assembly 132 can include the torque shaft 136 and the rotor 140 discussed above. The torque shaft 136 can be disposed in substantially the center of the expandable housing 155, e.g., equally spaced from an inside surface of the stent body 156 and can extend from the first end 148 disposed adjacent to the motor 112 toward the second end 152 of the expandable housing 155. The rotor 140 can be rotatably coupled to the motor 112 near the first end 148 of the expandable housing 155. The torque shaft 136 can be coupled to the rotor 140 such that as the rotor 140 rotates so does the torque shaft 136. The rotor 140 can be directly coupled to an output shaft of the motor 112 or can be rotated by magnetic fields generated by the motor, e.g., in a plurality of windings 114 of the motor 112.

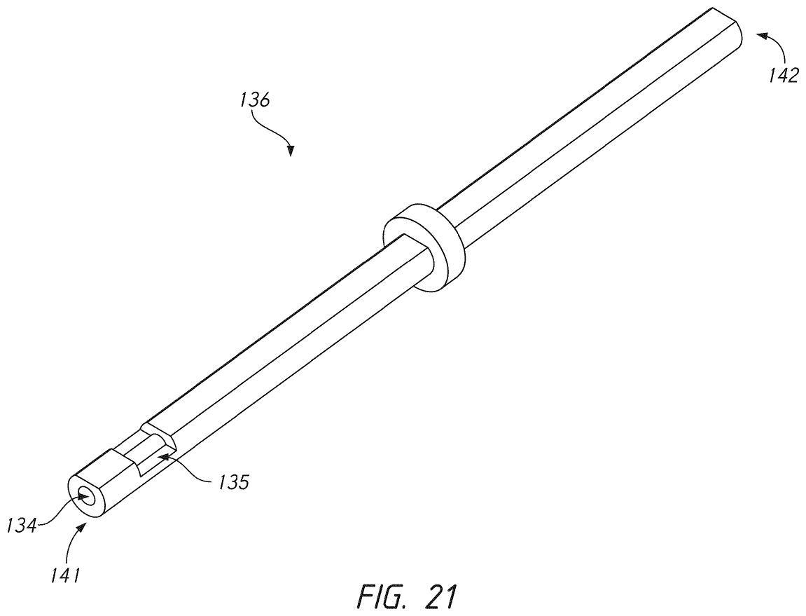

As shown in FIG. 21, the torque shaft 136 can include a recess 134 and an opening 135. The recess 134 can extend along the length of the torque shaft 136 from a first end 141 of the torque shaft 136. The recess 134 can extend from the first end 141 toward a second end 142 of the torque shaft 136 opposite the first end 141. The recess 134 could but need not extend along the entire length of the torque shaft. The recess 134 can extend a distance through the torque shaft 136 to a positioned between the first end 141 and the second end 142. The recess 134 can extend a distance through the torque shaft 136 of about 5 mm, about 10 mm, about 15 mm, about 20 mm, about 25 mm, about 30 mm, and/or any values between the aforementioned values. The recess 134 can be configured to receive the rotor 140 or a portion thereof (e.g., a shaft). The opening 135 can extend from an outer surface 139 of the torque shaft 136 to the recess 134. The opening 135 can be positioned a distance from the first end 141 of the torque shaft 136. The recess 134 can be exposed along the outer surface 139, e.g., at the opening 135, to provide access to the rotor 140 or the portion thereof positioned in the recess 134 so the rotor 140 or the portion thereof can be coupled to the torque shaft 136. For example, the rotor 140 or the portion thereof can be welded or soldered to the torque shaft 136. Accordingly, when the rotor 140 and/or the motor 112 rotates, the rotor 140 or the portion thereof can rotate the torque shaft 136.

The first end 168 of the impeller blade frame 164 can be coupled to the torque shaft 136 directly or by way of the hub 121 as discussed above. The first end 168 can be disposed at or adjacent to the first end 148 of the expandable housing 155 when the impeller 108 is disposed in the expandable housing 155. The tensile structure 176 can form a surface area between the torque shaft 136, the inner edge of the impeller blade frame 164 and between the first end 168 and the second end 172 of the impeller blade frame.

The power lead 252 of the system for chronic support 100 can include a distal end 256 and a proximal end 260 opposite the distal end 256. The distal end 256 can be coupled to an end of the motor 112 opposite the first end 148 of the housing 144. The distal end 256 of the power lead 252 can be coupled with a proximal part of the housing 144. The power lead 252 can be configured to supply power to the motor 112. The proximal end 260 can be coupled to or form part of a power assembly 268.

A circumferentially narrowed section 149 of the housing 144 can be disposed between the connection point of the distal end 256 of the power lead 252 and the portion of the housing 144 disposed around the motor 112. The circumferentially narrowed section 149 can form part of a delivery or a retrieval system. The circumferentially narrowed section 149 allows an instrument to grasp the housing 144 whereby a tension force or a compression force can be applied to aid in removal or delivery of the system for chronic support 100 from a patient. A delivery system can also apply a distal load to hold the fluid pump 104 steady while a sheath is withdrawn, as discussed further below.

The power assembly 268 can comprise a power supply device 276 and an energy source 278. The power supply device 276 can be implanted under a patient's skin 62 and the energy source 278 can transfer energy to the power supply device 276 from outside the patient's skin 62. The power supply device 276 can supply current to the motor 112 through a current conductor 240 within the power lead 252. In some embodiments, the energy source 278 that can generate a magnetic field in a first coil of the energy source and the power supply device 276 can include a second coil that can convey a current to the current conductor 204. The second coil can be mounted in or supported by a support member 280. The support member 280 can be a flat or planar structure. The support member 280 can be a flexible surface member that can encapsulate the coil. The support member 280 can be configured to be placed under the skin to remain in a relatively stable position under the skin.

In some embodiments, the fluid pump 104 can be in a powered state when the power supply device 276 transfers current to the motor 112. When the fluid pump is in a powered state, the motor 112 can cause the rotor 140 to rotate. The rotor 140 can cause the torque shaft 136 to rotate, which can cause the impeller 108 to rotate. The rotor 140, the torque shaft 136, and the impeller 108 can rotate about the central axis 106 of the fluid pump 104. The impeller 108 can pump or push fluid as the impeller 108 rotates about the central axis 106. The angle 184 between the radial direction of the first end 188 of the impeller 108 and the radial direction of the second end 192 of the impeller 108 can be configured to cause the impeller 108 to pump or push blood or fluid proximally in the direction of the central axis 106. In these embodiments, the impeller 108 can rotate about the central axis 106 in a first direction. The first direction can be a direction that causes the second end 172 to be the leading edge of the impeller blade 124. The first direction can be counterclockwise as viewed from the proximal end (as in FIG. 13A).

In some embodiments, the fluid pump 104 can be in an unpowered stated when the power supply device 276 does not transfer current to the motor 112. In some cases, when the fluid pump 104 is in the unpowered state, the angle 184 can allow blood or fluid flow through the pump relatively unimpeded. In this way, the impeller 108 can be non-obstructive to at least some blood flowing between the impeller blades 124 when the impeller 108 is not rotating. In some cases when the fluid pump 104 is in the unpowered state, the rotor 140 can be configured to rotate freely about the central axis 106. In these embodiments, fluid flow through the fluid pump 104 can apply a force to the impeller 108, causing the impeller 108 and the rotor 140 to rotate. The rotation of the rotor 140 can cause windings 114 of the motor 112 to generate electricity. More generally, the windings 114 can be one component of a coil assembly 204 that can be disposed in the housing 144. In some embodiments, the generated electricity can be directed through conductors of the coil assembly 204 to a battery 196 disposed in the housing 144, e.g., adjacent to the motor 112. In some embodiments, the generated energy can be transferred through the conductor 240 in the power lead 252 to the power supply device 276. In these embodiments, the power supply device 276 can be configured to store energy. Depending on the direction of natural flow, the impeller 108 can rotate about the central axis 106 in the first or in a second direction. In some embodiments, the second direction can be opposite the first direction. In some embodiments, the second direction can be the same direction as the first direction. In some embodiments, the blood flow can cause rotation in the same direction as when the fluid pump 104 is in the powered state, but the tensile structure 176 can be oppositely loaded. That is, in the powered state the blood can be pushed by first side of the tensile structure 176 of an impeller blade 124 and in the unpowered state the blood can push on the opposite side of the tensile structure 176 of the impeller blade 124. This behavior can result when the pump is placed such that the second end 152 of the expandable housing 155 is upstream of the first end 148. The rotational direction of the impeller 108 can be reversed in the unpowered state if the second end 152 of the expandable housing 155 is positioned downstream of the first end 148.

In some embodiments, the fluid pump 104 can be provided with an input for physiological conditions, for example heart rate, blood pressure, and/or cardiac output. In some embodiments, the fluid pump 104 can be switched between a powered state and an unpowered state based on the physiological conditions. In some embodiments, the fluid pump 104 can be in a powered state when a heart rate is high, e.g., at or above about 80 beats per minute, and the fluid pump 104 can be in an unpowered state when the heart rate is low, e.g., less than about 80 beats per minute. In the low heart rate condition, the small angle 184 can allow the presence of the unpowered fluid pump 104 not to impede or to minimally impeded the work of the heart in pumping blood. In the low heart rate condition, the impeller blade 124 can be allowed to rotate by the blood whereby energy can be harvested from the patient to store energy in a battery 196 as discussed further below.

In one embodiment, the input for physiological conditions can be the fluid pump 104. The fluid pump 104 can be periodically switched to an unpowered state. In this embodiment, the impeller 108 can freely rotate when the fluid pump 104 is in an unpowered state. Blood flow through the fluid pump 104 can cause the impeller 108 and consequently the rotor 140 to rotate. Rotation of the rotor 140 can cause the motor 112 to generate an electrical signal, e.g., a current in windings 114 of the motor 112. The fluid pump 104 can convert or translate the generated current or other signal into a value corresponding to native cardiac output, native blood flow at the location of the fluid pump 104 or another relevant physiological variable. The fluid pump 104 can use the estimate of native cardiac output to automatically adjust how fast the impeller 108 spins when the fluid pump 104 is in a powered state. For example, it can be desirable to increase the natural fluid flow rate by 33 percent as one example. The fluid pump 104 can detect the native cardiac output and automatically increase or decrease how fast the impeller 108 spins such that the native flow at the location of the pump is 33 percent greater when the fluid pump 104 is in the powered state than when it is in the unpowered state, e.g., increasing a native flow of 3 liters per minute to 4 liters per minute. By sensing cardiac output and controlling additional flow generated by the fluid pump 104 undesirable over-pumping, which could lead to depriving other organs served by the vasculature, can be avoided.

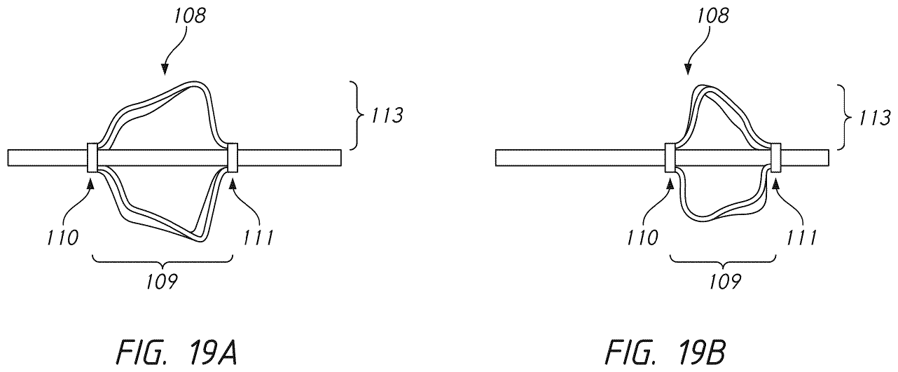

FIGS. 19A and 19B show additional aspects of embodiments of the impeller 108 and variations thereof. The impeller 108 can include a length 109 between a distal end 110 and a proximal end 111. The impeller 108 can include a length 109 of about 10 mm, about 12 mm, about 14 mm, about 15 mm, about 16 mm, about 18 mm, about 20 mm, about 22 mm, about 24 mm, about 25 mm, about 26 mm, about 28 mm, about 30 mm, about 32 mm, about 34 mm, about 35 mm, about 36 mm, about 38 mm, about 40 mm, about 42 mm, about 44 mm, about 45 mm, about 46 mm, about 48 mm, about 50 mm, and/or any value between the aforementioned values.

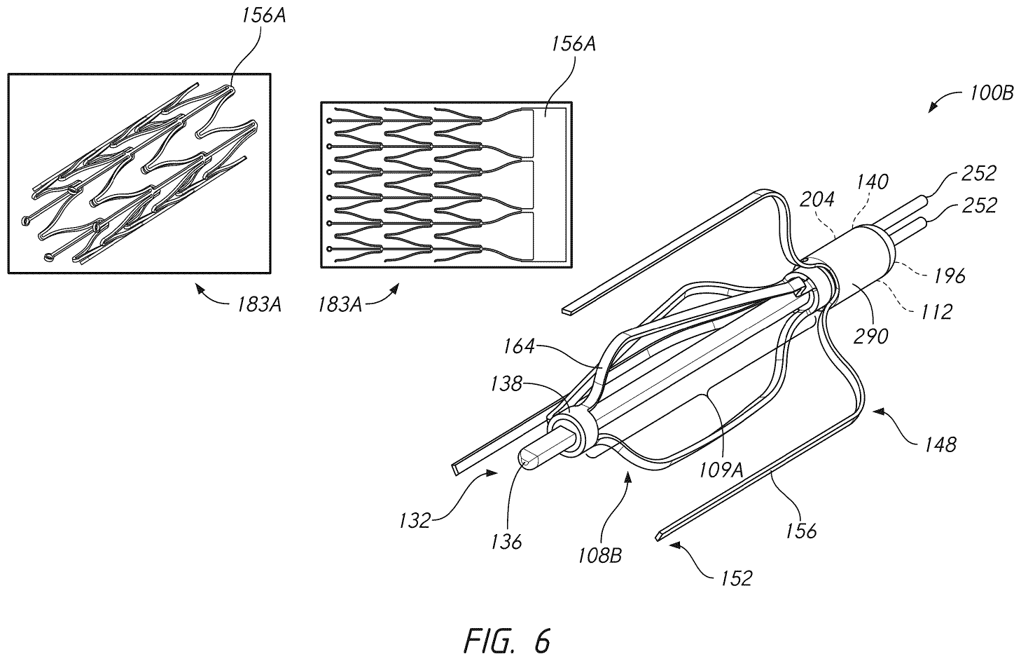

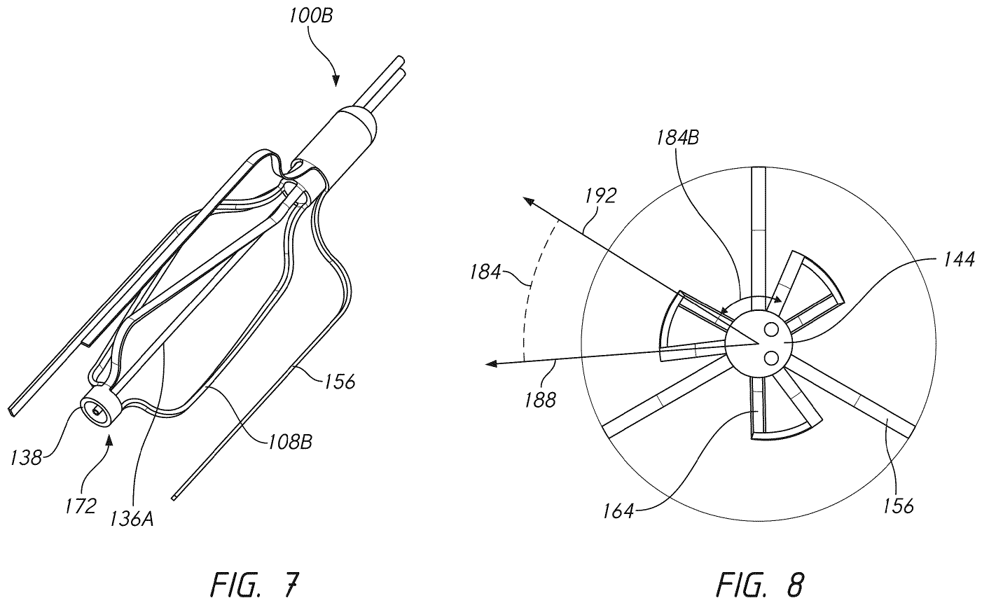

As shown in FIGS. 19A and 19B, the length 109 of the impeller 108 can be shortened or reduced to modify a ratio between the length 109 of the impeller 108 and a width 113 of the impeller 108 (i.e., the radial dimension). The impeller 108 can include a ratio between the length 108 and the width 113 of about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, and/or any value between the aforementioned values. For example, the impeller 108A, as shown in FIGS. 6-7, can include a length 109A of about 40 mm, the impeller 108, as shown in FIG. 19A can include a length 109 of about 24 mm, and the impeller 108, as shown in FIG. 19B can include a length 109 of about 18 mm.

The length 109 of the impeller 108 or the ratio between the length 109 and the width 113 can be modified based on the motor 112, a particular RPM of the impeller 108, a particular flow rate generated by the impeller 108, and/or a particular fluid pressure in the blood vessel of the patient. An impeller 108A with a length 109A, for example as shown in FIGS. 6-7, can comprise blades with a larger surface area than an impeller 108 with a length 109, as shown in FIGS. 19A-19B. For example, the impeller 108A shown in FIGS. 6-7 with a length 109A of about 40 mm can generate a same flow rate or fluid pressure as an impeller 108 shown in FIG. 19A with a length 109 of about 24 mm, when the impeller 108A rotates at a lower RPM than the impeller 108. Additionally, an impeller 108A with a larger surface area can require more torque to rotate than an impeller 108 with a smaller surface area. Accordingly, if the motor 112 is configured to generate low torque and spin at high speeds, the impeller 108 with the smaller surface area can be used, and if the motor 112 is configured to generate high torque by spin at lower speeds, the impeller 108A with the larger surface area can be used. Therefore, an impeller 108A with a larger surface area can reduce or eliminate shearing of red blood cells (i.e., hemolysis).

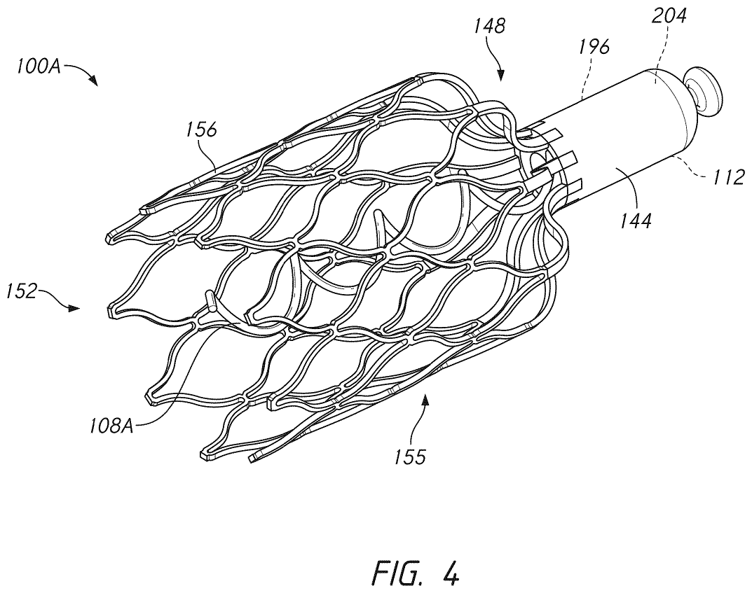

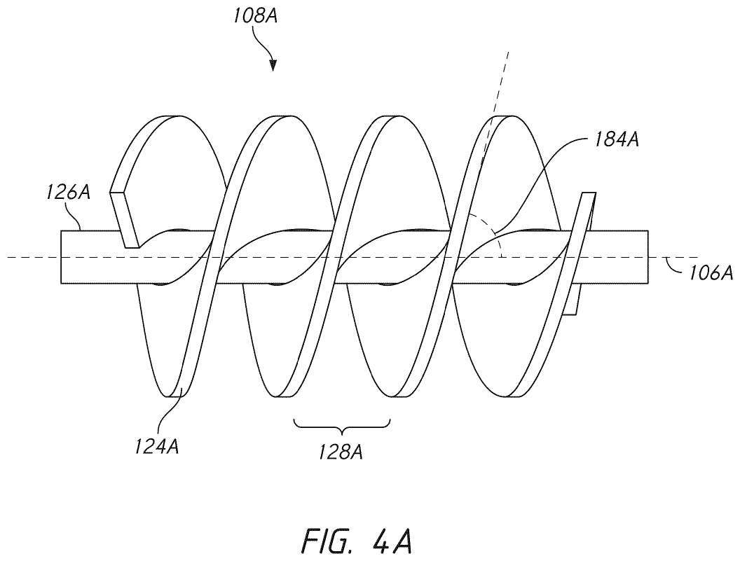

As discussed above, FIGS. 13-13A show that the impeller 108 can include a plurality of low angle blades. FIG. 4 shows an alternative embodiment 100A of the system for chronic support 100. Common features between the system for chronic support 100 and the system for chronic support 100A will not be described again but are incorporated here in their entirety. The system for chronic support 100A can include an alternative impeller 108A. Impeller 108A can comprise a screw, spiral or coil shape. In some embodiments, the impeller 108A can be an Archimedes screw, as shown in FIG. 4A. The screw, spiral or coil shape can be mounted at a proximal end to the motor 112 and can have a self-supporting free end. The impeller 108A can comprise a single helical impeller with a turn that extends about an angle greater than angle 184, e.g., more than 180 degrees, up to 360 degrees, in some cases two or more turns, in various embodiments more than ten turns and up to 20 or 30 turns in various embodiments.

FIG. 4A shows that the impeller 108A can include a central hub member 126A and an impeller blade 124A with a screw shape (i.e., a helical surface surrounding the central hub member 126A). More than one impeller blade 124A can be provided. The impeller blade 124A can extend radially from a central hub member 126A. The impeller blade 122A can circumferentially wrap (e.g., turn) around the central hub member 126A at an angle 184A from a central axis 160A of the impeller 124A, such that each time the impeller blade 124A wraps around the central hub member 126A, the impeller blade 124A is separated by a distance 128A in the direction of the central axis 106A. The impeller blade 124A can wrap around the central hub member 126A one time, two times, ten times, and up to 20 or 30 times in various embodiments. The central hub member 126A can be or can be coupled to the torque shaft 136 such that when the torque shaft 136 rotates, the central hub member 126A and the impeller blade 124A rotate.

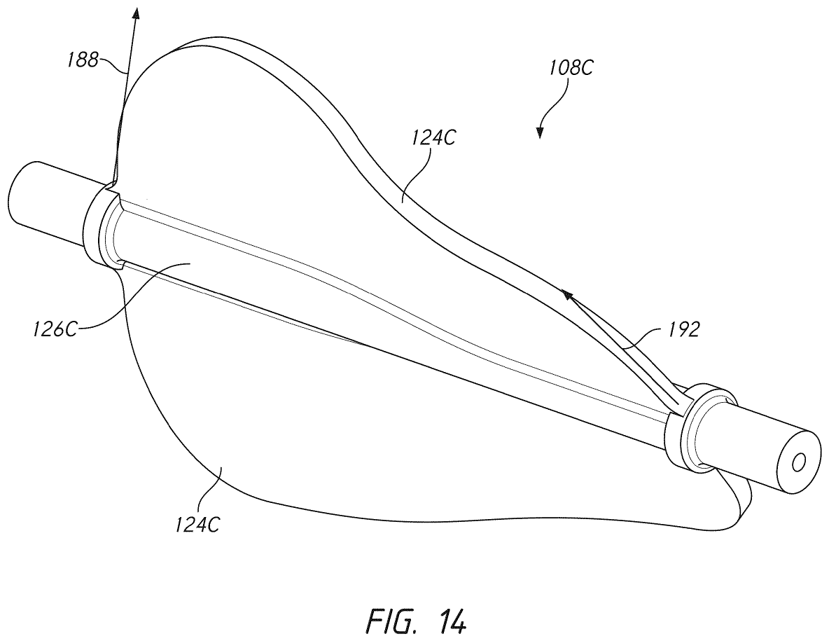

FIG. 14 shows another embodiment of an impeller 108C with a small angle blade. Common features between the impeller 108 and the impeller 108C will not be described again but are incorporated here in their entirety. In this embodiment, the impeller 108C can comprise two of impeller blades 124C. The impeller blade 124C can constitute a pair of blades. More than two impeller blades 124C or a single blade could be provided. An angle between the impeller blades 124C can be about 180 degrees. For example, the distal end of each impeller blade 124C can emerge from a central body or portion of the impeller 108C at a circumferential angle of 180 degrees from each other. The central body or portion of the impeller 108C can include a central hub member 126C. The central hub member 126C can be or can be coupled to the torque shaft 136 such that when the torque shaft 136 rotates, the central hub member 126C and the impeller blades 124C rotate. The impeller blades 124C can coil, fold, or furl around the central hub member 126C when the system for chronic support 100 is in the crimped state. In some configurations the central hub member 126C can be a fixed length and the coiled, folded or furled state can be achieved without axial elongation of the impeller 108C. In some configurations, the central hub member 126 can expand, e.g., be axially extended, or contract along the axis of the torque shaft 136 when the system for chronic support 100 is in a crimped state.

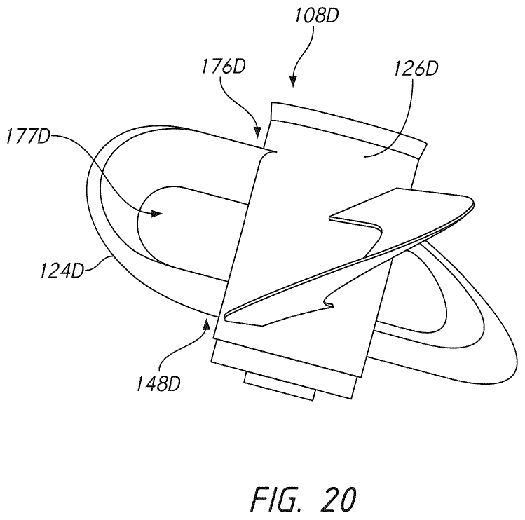

FIG. 20 shows another embodiments of an impeller 108D with a toroidal configuration. Common features between the impeller 108 and the impeller 108D will not be described again but are incorporated here in their entirety. In this embodiment, the impeller 108D can comprise impeller blades 124D. The impeller blades 124D can be ring shaped. The impeller blades 124D can extend radially from a central hub member 126D at a first end 148D of the impeller 108D. The impeller blades 124D can curve back towards the central hub member 126D and can be coupled to the central hub member 126D at a second end 176D of the impeller 108D. The impeller blades 124D can form a gap 177D between the impeller blades 124D and the central hub member 126D between the first end 148D and the second end 176D. The impeller blades 124D can be formed by cutting a sheet or tube of nitinol or other suitable metal and shaping the cut sheet or tube into the impeller blades 124D with the toroidal configuration. The toroidal configuration of the impeller 108D can reduce shearing forces applied to blood or fluid by the impeller blades 124D.

FIGS. 3, 11, and 12 show that the stent body 156 can be configured to secure the system for chronic support 100 to a wall of a blood vessel 54. The stent body 156 can apply an outward force on the wall of the blood vessel 54 creating mechanical engagement, e.g., tissue-structure overlap in the radial direction, a friction force between the stent body 156 and the wall of the blood vessel 54, or other manner of securement. The stent body can comprise a plurality of cells 158 bounded by a plurality of apices 159. The cells 158 can be holes in the stent body 156. The cells 158 can expand to a configuration against the wall of the blood vessel 54. The expandable housing 155 can be tapered portion, e.g., along a proximal portion such that the stent body 156 is spaced away from the wall of the blood vessel 54 at that portion. The cells 158 can allow blood to flow through the fluid pump 104 in that portion when the fluid pump 104 is in the blood vessel 54. FIG. 12 shows that in one embodiment a pattern of cells 158 can be generally regular sized and spaced diamond shaped openings. In one variation, the size of the diamond shaped openings can vary between the first end 148 and the second end 152 of the expandable housing 155.

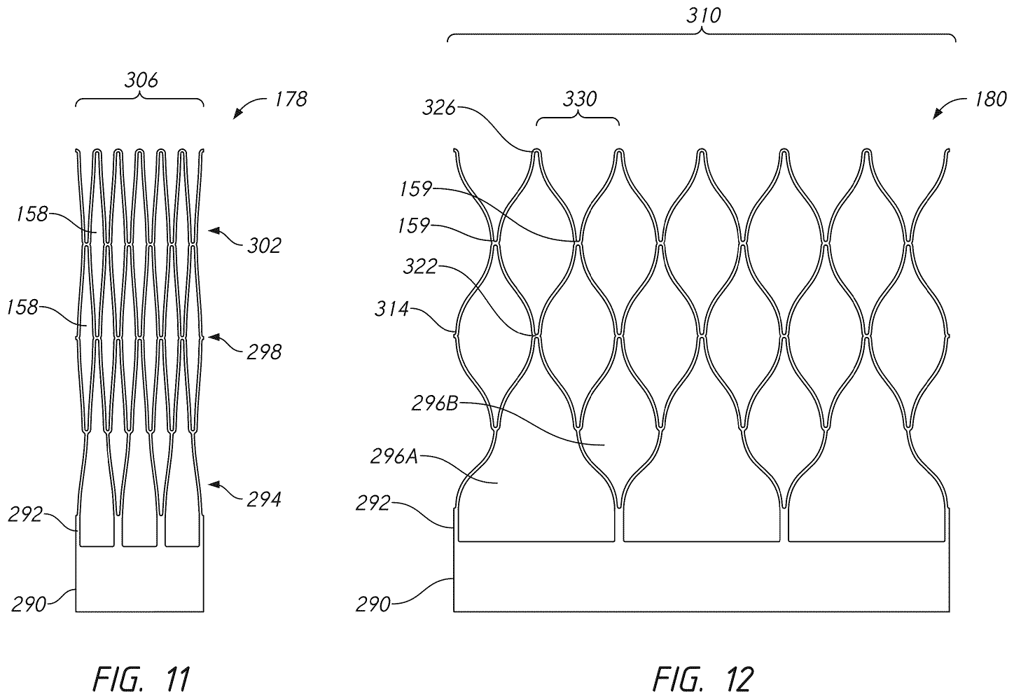

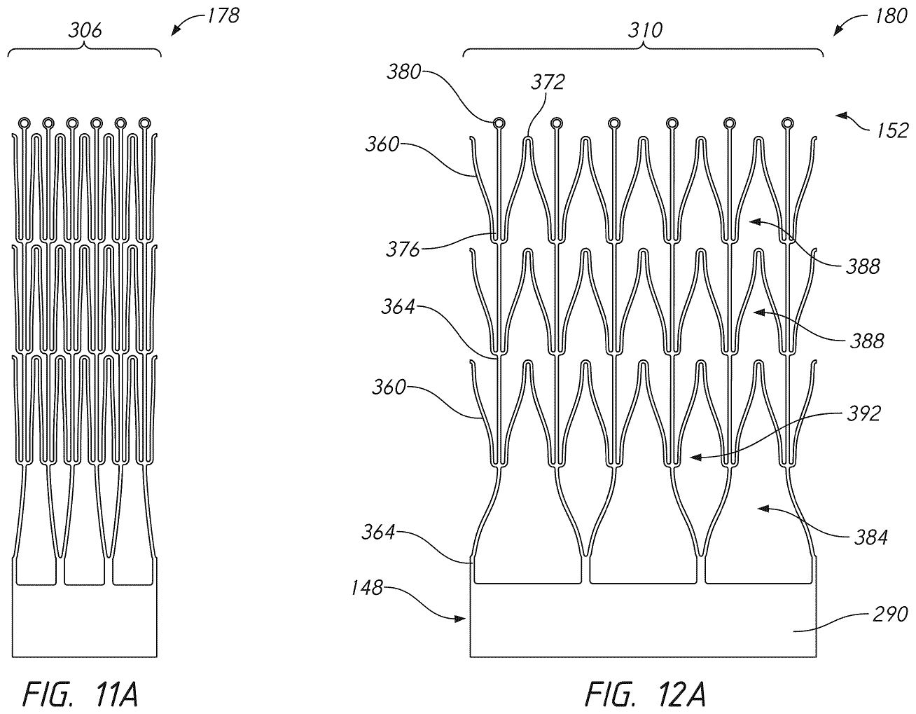

FIGS. 11 and 12 show the stent body 156 of the expandable housing 155 in a flat configuration. FIG. 11 shows a crimped state 178 of the flat configuration. FIG. 12 shows an expanded state 180 of the flat configuration. These figures also illustrate how the expandable housing 155 can be formed. The stent body 156 can be formed by cutting a sheet of nitinol or other suitable metal. The stent body 156 can include a cylindrical portion 290 that in the flat configuration has a rectangular shape. The flat form is later rolled into a cylinder and the cylindrical portion 290 forms a cylindrical body that can be secured to or disposed around the housing 144 as shown in FIG. 10. The expandable housing 155 includes a plurality of axial connectors 292 that extend from one side of the cylindrical portion 290 toward a fenestrated pattern of cells. The pattern of cells is laid out in a proximal cell row 294, an intermediate cell row 298, and a distal cell row 302. At least the intermediate cell row 298 and the distal cell row 302 includes the cells 158. The cells 158 can have a diamond shape when crimped and an enlarged diamond shape when expanded. The proximal cell row 294 can also have the cells 158. In some embodiments, the proximal cell row 294 has a plurality of enlarged cells 296A and a plurality of intervening cells 296B. The cells 158 can be formed between adjacent axial members 314. The axial members 314 can have an undulating pattern, e.g., a sinusoidal from in a proximal-distal direction. Adjacent axial members 314 can be joined at proximal and distal ends of each cell 158 forming the apices 159. Adjacent axial members 314 can be joined at circumferential ends of each cell 158 at a mid-cell connection 322. The enlarged cells 296A can be formed between the cylindrical portion 290 and proximally facing edges of the intermediate cell row 298. The enlarged cells 296A can be formed between connection of the axial members 314 to the axial connectors 292 proximal of the proximal apices 159 of the intermediate cell row 298.

The stent body 156 can be configured to not be separately supported at the distal end of the distal cell row 302. The cells 158 can have free apices 326 that are not separately supported. The stent body 156 can have a crimped circumference 306 in the crimped state and an expanded circumference 310 in the expanded state. In the expanded state adjacent free apices 326 can be spaced apart by a free apex spacing 330. A system for chronic support 100A includes a different configuration of a stent body 156A. The stent body 156A is similar in several aspects to the stent body 156, as discussed below.

FIGS. 3A-3B show details and variations of the power assembly 268. FIG. 3A shows one embodiment of a power assembly 268A that uses ultrasound to transfer energy through the skin 62 of the patient 50. The power assembly 268A can include a first transducer 276A and a second transducer 278A. In this embodiment, the first transducer 276A can be implanted in or under the skin 62 of a patient, and the second transducer 278A can be coupled to a surface of the skin 62 of the patient. In some embodiments, the second transducer 278A can be coupled to the patient's skin 62 with a coupling gel 282. The coupling gel 282 can significantly reduce or eliminate air between the second transducer 278A and the skin 62, ensuring that sound waves are able to pass from the second transducer 278A through the skin 62. The second transducer 278A can be coupled to a power supply 286. The power supply 286 can supply a current to the second transducer 278A. The second transducer 278A can convert the current into sound waves. The sound waves can travel through the coupling gel 282 and the skin 62. The first transducer 276A can receive the sound waves and convert the sounds waves to current in the conductor 240 to supply power to the fluid pump 104. In some embodiments, sound waves generated by the body of the patient 50 or other sources travel to the first transducer 276A and the first transducer 276A can convert the sound waves into current in the conductor 240.

In some embodiments, energy can be transmitted by a manner other than sound waves. For example, the first transducer 276A and the second transducer 278A can be induction coils. The second transducer 278A can be configured to generate magnetic fields when a current is applied to the second transducer 278A. The magnetic fields can travel through the skin 62 of the patient. The first transducer 276A can respond to the magnetic fields by generating current in response to the magnetic fields to deliver the current in the conductor 240.

In another variation, the second transducer 278A can be configured to generate infrared radiation when a current in applied to the second transducer 278A. The second transducer 278A configured to generate infrared radiation can be located remote of the skin 62, e.g., need not be generated by a device that touches the skin. The infrared radiation can travel through the skin 62. The first transducer 276A can receive the infrared radiation and convert the infrared radiation to current in the conductor 240.

In another embodiment, the second transducer 278A can be configured to generate millimeter waves when a current is applied to the second transducer 278A. The second transducer 278A configured to generate millimeter waves can be located remote of the skin 62, e.g., need not be generated by a device that touches the skin. The millimeter waves can travel through the skin 62. The first transducer 276A can receive the millimeter waves and convert the millimeter waves to current in the conductor 240.

In some embodiments, the second transducer 278A can be configured to generate radio waves in a wavelength of 20-300 GHz. The second transducer 278A configured to generate radio waves can be located remote of the skin 62, e.g., need not be generated by a device that touches the skin. The radio waves mar travel through the patient's skin 62. The first transducer 276A can receive the radio waves and convert the radio waves to current in the conductor 240.

In some embodiments, the power assembly 268 can be configured to transmit energy through a thickness extending from the second transducer 278A to a depth of the first transducer 728B. In some embodiments, the thickness can comprise a depth of about 1 mm, about 5 mm, about 10 mm, about 15 mm, about 20 mm, about 24 mm, about 25 mm, about 30 mm, about 35 mm, about 36 mm, about 40 mm, about 45 mm, about 48 mm, about 50 mm, and/or any value between the aforementioned values. In some embodiments, the thickness can include a depth between about 5 mm and about 50 mm. In some embodiments, the thickness can include a depth between about 10 mm and about 40 mm. In some embodiments, the thickness can include a depth between about 20 mm and about 30 mm.

FIG. 3B shows that in some embodiments, a tattoo or dermal sticker can be placed on, in or under the skin 62. The tattoo can comprise conductive wire, sheet, or ink. The tattoo can present a target printed or placed on the skin 62. The tattoo can be configured to enhance transmission of energy through the patient's skin 62. The tattoo can be used as a target for aiming energy sources and/or as part of a circuit for generating current. For example, a power supply 286 can power a second transducer 278A that is located remote from the skin. The second transducer 278A can be configured to transmit infrared, millimeter wave, radio waves or other energy capable of remote transmission to the tattoo. The tattoo can be configured to either generate a current in response to the energy being directed thereto or can merely act as a target such that the energy is received efficiently at the first transducer 276A. The first transducer 276A can then generate current in the conductor 240 to the fluid pump 104.

Having described the system for chronic support 100 in various embodiments, some methods of use and additional systems will be addressed. FIGS. 1-2B show that the fluid pump 104 can be placed at various locations in the patient 50. In some embodiments, the fluid pump 104 can be placed such that when the fluid pump 104 is in a powered state, the rotation of the impeller 108 in the first direction can pump or push blood or fluid in a direction of a natural blood or fluid flow. In some embodiments, the fluid pump 104 can pump or push blood or fluid at a flow rate up to 5 L/min. FIG. 2 shows that the fluid pump 104 can be placed in an artery Ay, e.g., the descending aorta to pump blood with native flow. The expandable housing 155 can be expanded such that the second end 152 is open to blood flow directly into the internal space where the impeller 108 (not shown) is located and operates when the motor 112 is operating. The proximal end of the housing 144 is located downstream of the open expandable housing 155. FIG. 2 shows that the fluid pump 104 can be placed in a vein Vn such that the open second end 152 of the expandable housing 155 is facing the direction of inward flow of blood in the venous vasculature. The blood flows directly onto the impeller 108 (not shown) and can be pumped thereby out of the expandable housing 155 to supplement native flow. The fluid pump 104 advantageously provides that the first structure of the pump that blood located in the central lumen of the blood vessel contacts is the impeller 108. This open-ended configuration reduces the number of strut or other structure crossings by roughly one-half by eliminating a distal tapered portion that would be present in a configuration that is symmetrical about a mid-transverse plane. Thus, FIG. 2 shows two placements of the fluid pump 104. In one application, a fluid pump 104 is placed in each of the artery Ay and the vein Vn and each pump is operated simultaneously or in a coordinated manner.

In other embodiments, the fluid pump 104 can be placed such that when the fluid pump 104 is in a powered state, the rotation of the impeller 108 can resist the natural blood or fluid flow. In one embodiment the fluid pump 104 can be placed in an aorta of the patient, e.g., adjacent to or within a renal artery. The impeller 108 of the fluid pump 104 could be operated to selectively resist flow downstream of the renal artery resulting in an increase of pressure at the renal artery ostium. In this embodiment, when the fluid pump 104 is in a powered state the rotation of the impeller 108 can increase blood flow through the renal artery, thereby increasing the flow into kidneys of the patient 50. The increased blood into the kidneys can increase diuresis. In another embodiment, the fluid pump 104 can be placed in a portion of a vena cava of the patient 50 (e.g., in the superior vena cava or in the inferior vena cava). In this embodiment, when the fluid pump 104 is in a powered state the rotation of the impeller 108 in the first direction can improve cardiac output. When the fluid pump 104 is in a powered state, the rotation of the impeller 108 in the first direction can generate outflow to resist native blood flow in the portion of the vena cava. In another embodiment, the fluid pump 104 can be placed adjacent to or in a renal vein of the patient 50, and when the fluid pump 104 is in a powered state, the rotation of the impeller 108 can decrease renal venous pressure. In this embodiment, the fluid pump 104 can enhance or improve blood flow through a kidney of the patient 50. In another embodiments, the fluid pump 104 can be placed in a portion of the vena cava of the patient 50 upstream from the outflow of the renal veins from the kidneys of the patient 50. The impeller 108 of the fluid pump 104 could be operated to resist flow from upstream of the renal veins in the vena cava which can result in a decrease of pressure at the renal vein ostia and/or in greater flow from the renal veins. In this embodiment, when the fluid pump 104 is in a powered state the rotation of the impeller 108 can increase pressure upstream from the fluid pump (i.e., towards the legs of the patient 50), and decrease pressure at the renal vein outflow into the vena cava. The increased pressure upstream from the fluid pump and the decreased pressure at the renal vein outflow can increase an amount of blood the heart pumps from the kidneys of the patient 50 and decrease the amount of blood the heart pumps from the legs of the patient 50.

In other embodiments, the fluid pump 104 can be placed adjacent to or within a thoracic duct of the patient 50. When the fluid pump 104 is in a powered state the rotation of the impeller 108 can modulate or regulate flow from the thoracic duct of the patient 50 to the blood vessel 54 of the patient 50. In one embodiment, the rotation of the impeller 108 in the first direction can increase a flow of fluid out of the thoracic duct, thereby increasing a flow of fluid in the thoracic duct. An increase of the fluid flow in the thoracic duct can reduce interstitial fluid pressure. In another embodiment, the rotation of the impeller 108 in the first direction can resist or impede the flow of fluid out of the thoracic duct. In this embodiment, the fluid pump 104 can reduce a fluid volume contribution from the thoracic duct to the volume of fluid in the vasculature.

In some configurations, multiple fluid pumps 104 can be placed in the patient 50. In one configuration, as shown in FIG. 2A, a first fluid pump 104 can be placed in the blood vessel 54 below or downstream from the kidneys, and a second fluid pump 104 can be placed in the blood vessel 54 above or upstream from the kidneys. The first fluid pump 104 can be in an unpowered state. In this way, mechanical resistance from the first fluid pump 104 can create an area of high pressure below the kidneys. Furthermore, the rotation of the impeller 108 of the first fluid pump 104 in the unpowered state can charge a battery 196, or the rotation of the impeller 108 of the first fluid pump 104 can generate a current directed to the second fluid pump 104 by the power lead 252 to directly power the second fluid pump 104. In these configurations, the second fluid pump 104 which is located higher in the descending aorta can be in a powered state, and the second fluid pump 104 can pump or push blood or fluid into the kidneys increasing blood or fluid flow into the kidneys, which can create an arca of high pressure above the kidneys. The area of high pressure above the kidneys, and the area of increased flow resistance due to the presence of the first fluid pump 104 below the kidneys can improve perfusion or diuresis. It is to be appreciated that the first fluid pump 104 and the second fluid pump 104 can be placed upstream of the kidneys while enhancing diuresis. For example, the second fluid pump 104 could power the first fluid pump 104 which is located downstream of the first fluid pump 104 and upstream of the renal arteries.

In another configuration, as shown in FIG. 2B, a first fluid pump 104 can be placed in an arterial location Ay and a second fluid pump 104 can be placed in a venous location Vn. The first fluid pump 104 can be in the unpowered state, and the rotation of the impeller 108 of the first fluid pump 104 in the unpowered state can charge a battery 196. The battery 196 can power the second fluid pump 104. Or the impeller 108 of the first fluid pump 104 can also be coupled to the second fluid pump 104 by the power lead 252 such that the first fluid pump 104 generates current to directly power the second fluid pump 104 in the venous location Vn. The system illustrated in FIG. 1B can be reversed, such that the impeller 108 of the second fluid pump 104 can be rotated by the blood with the second fluid pump 104 in an unpowered state. The rotation of the impeller 108 can create current for directly powering the first fluid pump 104 or for storing energy in the battery 196 for later use by the first fluid pump 104 in the venous location or the second fluid pump 104 in the arterial location.

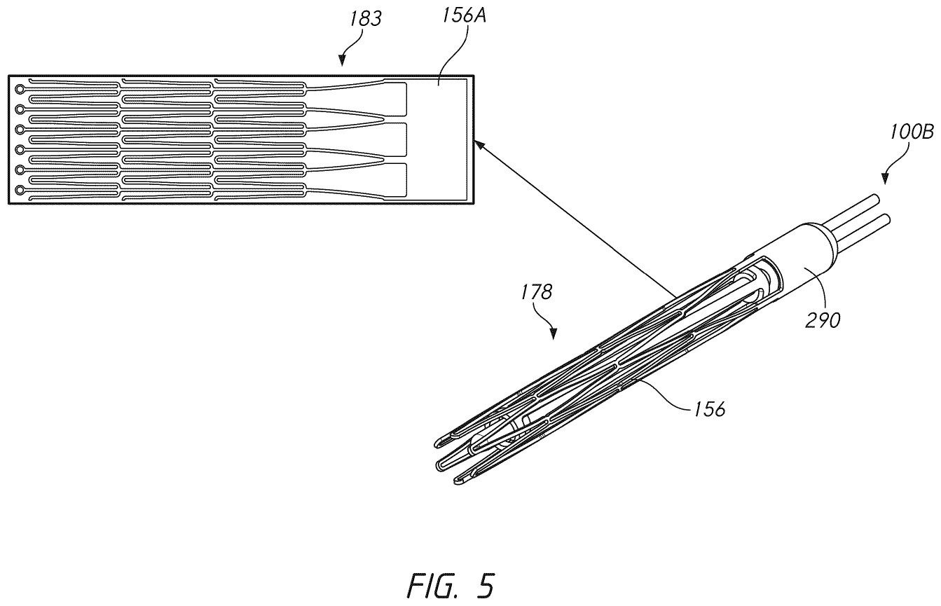

FIGS. 5-8, 11A, and 12A show an alternative embodiment 100B of the system for chronic support 100. Common features between the system for chronic support 100 and the system for chronic support 100B will not be described again but are incorporated here in their entirety. In this embodiment, a portion of the stent body 156 can cover the housing 144. The system for chronic support 100B can be expandable. A simplified stent body 156 is shown in FIGS. 5 and 6 for illustrative purposes only. Inset images 183 and images 183A show details of an alternative stent body 156A in the crimped state 178 and the expanded state 180 respectively, as discussed below with reference to FIGS. 11A and 12A. FIG. 5 shows the system for chronic support 100B in a crimped state 178 and FIG. 6 shows the 100B in an expanded state 180. When the system for chronic support 100B is in the crimped state 178 the stent body 156 and an alternative embodiment of an impeller 108B, also shown in FIGS. 15C-15F, can be compressed against or closer toward the torque shaft 136 such that a diameter of the system for chronic support 100B is less that the diameter of the impeller 108B when the impeller 108B is in the expanded state 180. These diameters are illustrated in the flat depiction of the stent body 156B in FIGS. 11A and 12A discussed below. The crimped state 178 can allow the system for chronic support 100B to move through the blood vessel 54 of the patient 50 more easily that when the system for chronic support 100B is in the expanded state 180.

In this embodiment, the system for chronic support 100B can comprise a torque shaft 136 that extends beyond the second end 152 of the impeller 108B. The impeller 108B can be coupled to the torque shaft 136 at the first end 148 and the second end 172. In this way, the torque shaft 136 can provide more radial stability to the second end 172 of the impeller 108B. The second end 172 of the impeller 108B can be slideable along the torque shaft 136 because the distance between the first end 148 and the second end 172 of the impeller 108B can longer when the system for chronic support 100B is in the crimped state 178 than when the system for chronic support 100B is in the expanded state 180. In this embodiment, the second end 172 of the impeller 108B can be slideably coupled to the torque shaft 136 via a ring 138. A diameter of the torque shaft 136 and a diameter of the ring 138 can be the same diameter. FIG. 6 shows that the ring 138 and the torque shaft 136 can have peripheries that include arcuate, e.g., circular segments. The ring 138 and the torque shaft 136 can have peripheries with segments that include flat portions to enhance torque transmission therebetween. The ring 138 and the torque shaft 136 can have peripheries with arcuate segments and flat segments. The ring 138 assures that radially movement of the second end 172 of the impeller 108B with respect to the torque shaft 136 is restricted or prevent. FIG. 7 is a simplified view of the system 100B with the torque shaft assembly 132 removed. The position of the torque shaft 136 is illustrated by axle 136A.

FIG. 8 shows a view of the system for chronic support 100B from the proximal end e.g., from the end where the housing 144 is located. The radial direction of the first end 188 and the radial direction of the second end 192 can form the angle 184. In some embodiments, the angle 184 can be less than 45 degrees. In other embodiments, the angle can be 30 degrees or less. As discussed above with reference to FIG. 3, the angle 184 of the impeller 108B can cause the impeller 108B to pump or push blood or fluid in the direction of the central axis 106. In some embodiments, the system for chronic support 100B can include an angle 184B between a radial direction of the first end 188 of a first impeller blade 124 and a radial direction of the second end 192 of a second impeller blade 124. In an area of the angle 184B the impeller 108B can be non-obstructive to at least some blood or fluid flowing between the first impeller blade 124 and the second impeller blade 124. In this way, if the system for chronic support 100B is in the unpowered state, if the impeller 108B is unable to rotate, the fluid pump 104 can minimally obstruct the flow of blood or fluid through the blood vessel 54 or peripheral vessel 58. In some embodiments, the angle 184B can be about 75 degrees to about expanded state 180 degrees.

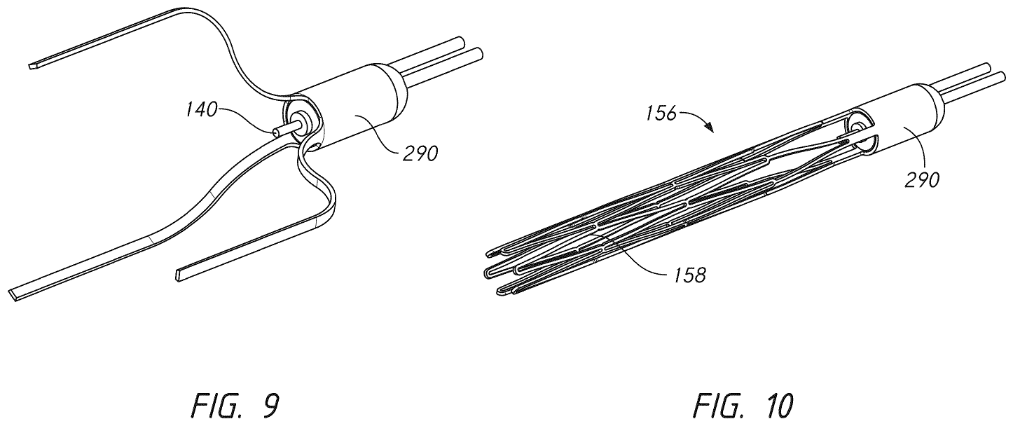

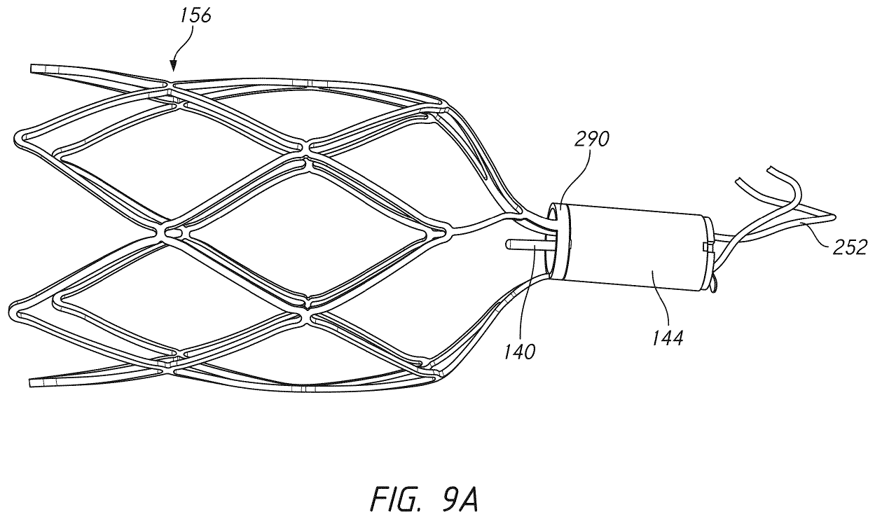

FIG. 9-10 show the system for chronic support 100B with the impeller 108B and the torque shaft 136 removed to simplify the view. In this embodiment, the rotor 140 can be disposed inside the housing 144, e.g., outside of the stent body 156. In this way, the torque shaft 136 can be completely disposed within the stent body 156. The torque shaft 136 can couple with an output shaft that extends out of the housing 144. Alternatively, the rotor 140 can be disposed within the stent body 156, outside of the housing 144. The rotor can be coupled the motor 112 through an output shaft or magnetic fields. FIG. 9 shows the cylindrical portion 290 of the expandable housing 155 disposed over and around the housing 144. FIG. 9A shows that the cylindrical portion 290 can be shortened and can be attached to a distal end of the housing 144 to reduce the stacking of layers over the motor 112. This can lower the profile of the assembly or allow a larger motor profile to be used without impacting profile significantly. In other variations the expandable housing 155 can be attached to the outside of the housing 144 (as in FIGS. 3, 15, 15B, and 16E) or the cylindrical portion 290 can form the motor housing. FIG. 10 shows the system for chronic support 100B in the crimped state 178 with the impeller 108 and the torque shaft 136 removed.

FIGS. 11A and 12A show the stent body 156A in a crimped state 178 and expanded state 180 respectively. As discussed above in connection with FIGS. 11 and 12, the stent body 156A can comprise an array of thin struts formed in any suitable manner, e.g., by laser cutting a sheet. FIGS. 11A and 12A show flat configurations that can be the laser cut pattern and also can illustrate features of the pattern. The stent body 156A can comprise a plurality of circumferential rings 360 from the first end 148 to the second end 152. The circumferential rings 360 can comprise a plurality of alternating peaks 372 and valleys 376. The valley 376 can be proximal-most portion of each circumferential rings 360. The peak 372 can be the distal-most portion of each circumferential rings 360. The circumferential rings 360 can be coupled together by a plurality of axial connectors 364 extending between the first end 148 and the second end 152, e.g., from the first end 148 to the second end 152, of the stent body 156A. The axial connectors 364 can be coupled to the circumferential rings 360 at each valley 376.

The circumferential rings 360 and the axial connectors 364 can form a border of a plurality of distal cells 388. Between a circumferential ring 360 located closest to the first end 148 and a cylindrical portion 290, the axial connectors 364 can form a plurality of alternating proximal cells 384 and transition cells 392. The proximal cells 384 can be formed by the axial connectors 364, the cylindrical portion 290 and the circumferential ring 360 located closest to the first end 148. The transition cells 392 can be formed by the axial connectors 364 and the circumferential ring 360 located closest to the first end 148. In some embodiments, the transition cells 392 can be substantially diamond shaped. The distal cells 388 can have an approximately chevron shape and can provide an even outward force along the cylindrical distal portion of the stent body 156A. The configuration of the circumferential rings 360 and the distal cells 388 provide for ease of self-expansion and may provide for a larger difference in size between the crimped state 178 and the expanded state 180 than other patterns, such as a uniform diamond pattern. Adjacent axial connectors 364 can merge into a merged axial connection portion for connecting the axial connectors 364 to the cylindrical portion 290.

In some configurations, the peaks 372 can be angled radially outward such that when the stent body 156A is in the expanded state 180 and the system for chronic support 100A is in a blood vessel 54, the peaks 372 can apply an outward force on the blood vessel 54 to provide active anchoring, such that the peaks 372 create pressure points that anchor the stent body 156A and the system for chronic support 100A in place. In some configurations, the axial connectors 364 can each include a circular nib 380 at the second end 152. The circular nibs 380 can provide additional active anchoring, however, the circular shape of the circular nibs 380 can be atraumatic, such that when the system for chronic support 100 is inserted into the patient 50, the circular nibs 380 do not injure or traumatize the wall of the blood vessel 54.

When the stent body 156A is in the crimped state 178, the stent body 156A can include a crimped circumference 306, and when the stent body 156A is in the expanded state 180, the stent body 156A can include an expanded circumference 310. The expanded circumference 310 can be larger than the crimped circumference 306. The cylindrical portion 290 can include the same circumference or diameter in the crimped state 178 and in the expanded state 180.

### Additional Configurations and Methods

FIGS. 15-15F show additional configurations of a system for cardiac support 400. The cardiac support system 400 can include many of the features discussed above and such features are incorporated to supplement the description that follows. The cardiac support system 400 includes a fluid pump 404 that has a proximal end 405 and a distal end 406. As is discussed above, the distal end 406 can operate as a fluid intake end if the blood is pumped distally to proximally. The distal end 406 can operate as a fluid outlet end if the blood is pumped proximally to distally. The fluid pump 404 can include a motor 424 that is coupled to a current source by a power lead 426. The current source can include a battery and/or an external source such as a system for transcutaneous energy.

The cardiac support system 400 can include a torque shaft assembly 432 that is coupled to the motor 424, e.g., to an output shaft, to a rotor, to a shaft extending from a rotor, etc., disposed in a housing 428 disposed around the motor 424. The torque shaft assembly 432 can include a torque shaft 436 that is cantilever supported within a volume defined by the internal surface of an expandable housing 440. The expandable housing 440 can include a stent portion that is expandable, e.g., that is self-expanding such as being formed of nitinol or other material that can have an expanded free state and can be elastically crimped or compressed. The stent portion and pattern can be similar to that of FIGS. 11A and 12A above. In the illustrated variation there are two circumferential rings 360 rather than three. However, the cardiac support system 400 can have a longer expandable housing 440 with three or more than three circumferential rings 360.

The expandable housing 440 can have a circumferentially narrowed section 444 that tapers down to the diameter of the housing 428. An array of filaments, e.g., a compressed circumferential ring or an array of axial members can be secured directly to an outside surface of the housing 428. This allows the cylindrical portion 290 of the pattern of FIGS. 11A and 12A to be omitted. Any suitable technique can be used to directly connect the ring or array of axial members to the housing 428, e.g., welding, an adhesive, or other suitable joining technique.

An impeller 408 is disposed on the torque shaft assembly 432, e.g., at least partially slideably disposed on the torque shaft 436. The impeller 408 includes a proximal ring 412 and a distal ring 416. A blade frame 420 extends between the rings 412, 416 similar to the structure discussed above in connection with FIGS. 6-8. In one configuration, the proximal ring 412 is slideable on the torque shaft 436 between a compressed state when the cardiac support system 400 is crimped and an expanded state when the cardiac support system 400 is capable of operating in a blood vessel.

A membrane or other tensile structure can be disposed between the blade frame 420 and the torque shaft 436 to provide for moving the blood upon rotation of the impeller 408 or for responding to pressure of the blood to rotate the impeller 408.

FIG. 15A shows a distal end view of the cardiac support system 400. The expanded impeller 408 can be seen to extend radially toward the inner side of the expandable housing 440. A small tip gap is provided therebetween. The impeller 408 includes three blades, though as discussed above other numbers of blades could be provided including two blades, four blades, five blades, six blades, seven blades, or eight or more blades. The blades can be aligned at a single axial position or can be arranged in two or more blade rows. In one configuration there are two blade rows, and the blades can rotate independently and even in opposite directions, as in a contra-rotating impeller. As discussed above, an angle 184 is provided between a radial direction of a first end 188 and a radial direction of a second end of the blade frame 420. The magnitude of the angle 184 is discussed above in connection with other variants of impellers discussed herein. The benefits of the small angle blades are also discussed above. The angle 184 is shown in more clarity in FIG. 15F, depicting the frame of the impeller 408 separate from the cardiac support system 400.

Although several embodiments disclosed herein have the structure seen n FIG. 15A, with an expandable housing 440 including a circumferentially narrowed section 444 transitioning to a cylindrical portion 448, a more complex geometry can be provided between the ends of the expandable housing 440. FIG. 15B shows that in some variations a waist portion 445 disposed between the circumferentially narrowed section 444 and a distal tapered portion 446. The waist portion waist portion 445 can provide enhanced stiffness compared to a cylindrical configuration. The segment proximal of the waist portion 445 can provide an enhanced anchoring effect by digging into the vessel wall tissue. The segment distal the waist portion 445 can be deflected by interaction with the vessel wall to provide or enhance the shaping of the vessel wall by the expandable housing. The distal tapered portion 446 is configured such that when placed in a blood vessel that is approximately the side of the waist portion 445, the circular nibs 380 are deflected to a greater extent resulting in more radially outward force. Greater radially outward force enhanced securement in the blood vessel by embedding the circular nibs 380 more deeply into the vessel wall.

FIGS. 15C-F show the cardiac support system 400 with the expandable housing 440 removed and the blade frame 420 and proximal and distal ring 412, ring 416 of the impeller 408. FIG. 15C shows that the torque shaft 436 is longer than the expanded distance between the proximal ring 412 and the distal ring 416. This additional length allows one or both of the proximal ring 412 and the distal ring 416 to translate along the torque shaft 436 as the impeller 408 is being expanded. As noted above, where the impeller 408 is formed from a frame member a tensile structure would be disposed about the torque shaft 436 and would be connected to each of the blade frame 420, e.g., to each of three blade frame 420.

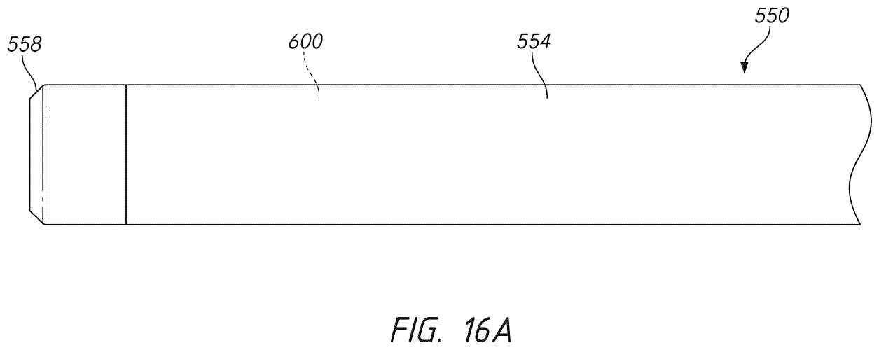

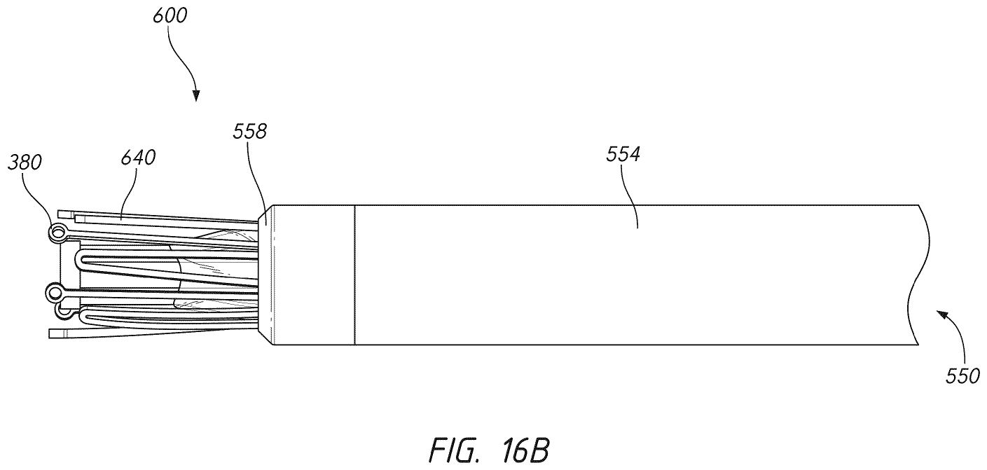

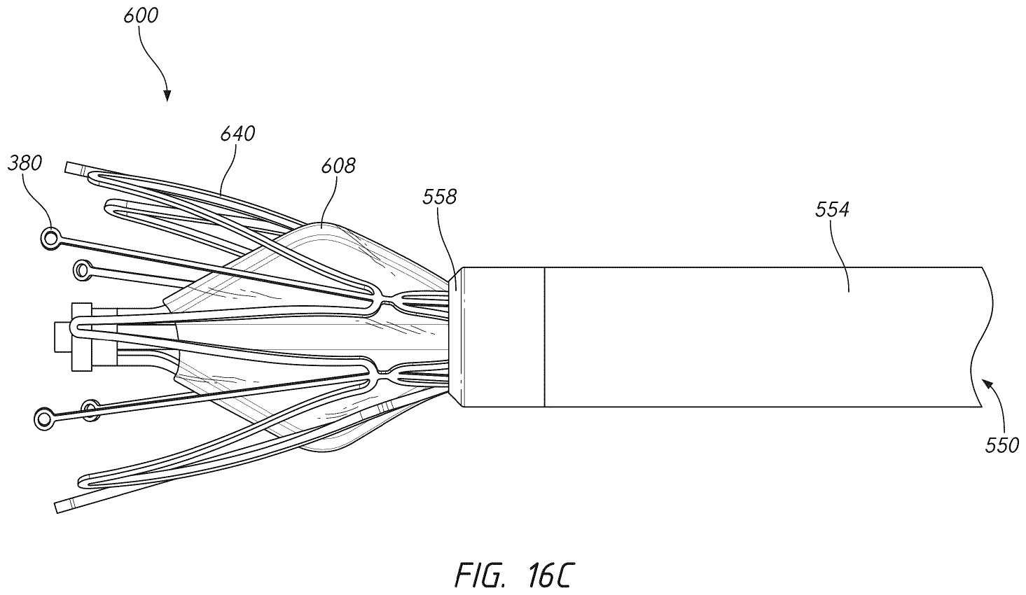

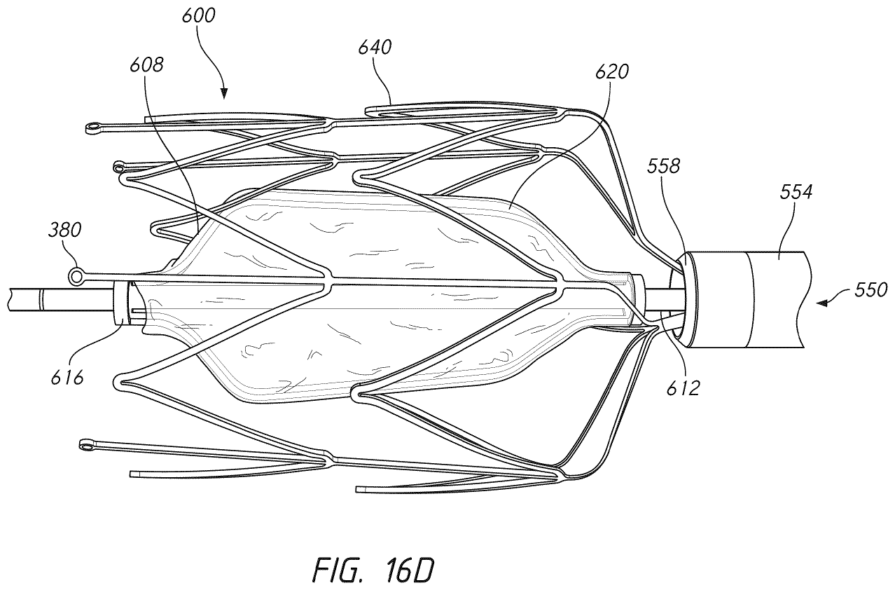

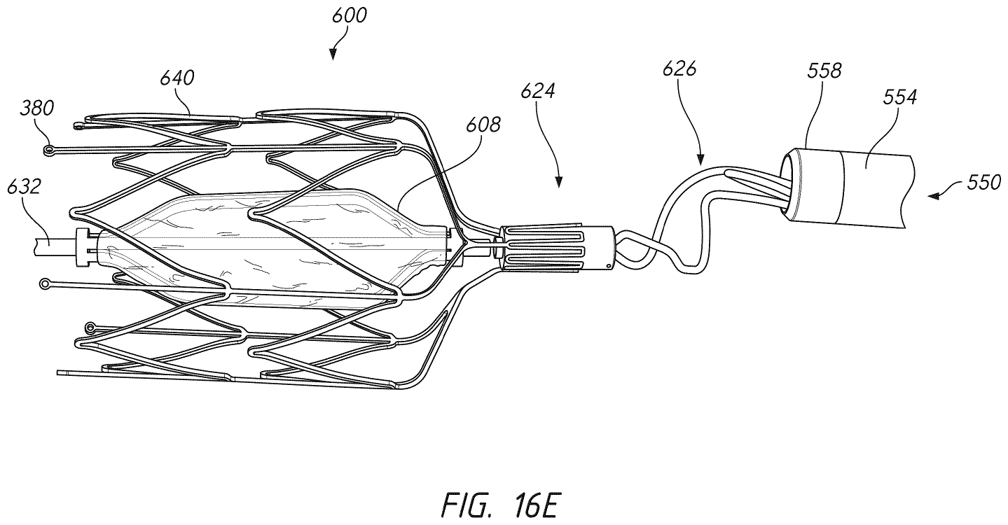

FIGS. 16A-16E illustrate another cardiac support system 600 as well as a delivery system 550 that can deploy the system 600. FIG. 16A shows a delivery system 550 that can include a sheath 554 extending from a proximal end (not shown) to a tip 558. The sheath 554 can be configured as a flexible elongate body that is sufficiently pushable and steerable to extend from a vascular access point (e.g., in a subclavian artery, a femoral artery, an iliac artery, a femoral vein, an iliac vein, a radial artery, a radial vein, etc.). The sheath 554 can have a radiopaque marker or a structure with greater or lesser stiffness than the proximal extent of the tip sheath 554. In use, the radiopaque marker or other structure enables visualization of the tip 558 at an appropriate position to provide for deployment of the cardiac support system 600.

After the tip 558 has reached an appropriate position, e.g., upstream of a renal artery, downstream of a renal vein or in another position useful for enhancing diuresis and/or supporting heart function in another way, the cardiac support system 600 can be deployed. The cardiac support system 600 can be held in a stationary position (e.g., by applying a load to tool interface). The load can be applied to an interface portion similar to the circumferentially narrowed section 149, discussed above, while the delivery system 550 is withdrawn to begin to expose a distal portion of the cardiac support system 600. FIG. 16B shows that the expandable housing 640 and initially the circular nibs 380 are exposed out of the tip 558. The expandable housing 640 may be held in the crimped state during initial extension of the cardiac support system 600 such that during this initial period the position of the cardiac support system 600 can be confirmed prior to full expansion.

FIG. 16C shows that further relative movement between the delivery system 550 and the cardiac support system 600 provides for extension of more of the expandable housing 640 distal of the tip 558. The circular nibs 380 and other distal aspects of the expandable housing 640 can begin to expand radially outwardly. Gradual expansion provided an angular profile to the partly expanded housing 640 so that the circular nibs 380 may touch down first on the vessel wall before the expandable housing 640 is fully released. This can provide a benefit of visually confirming the position of the cardiac support system 600 before full deployment which could allow for recapture and repositioning of the cardiac support system 600. This approach can also allow the vessel wall to act against the circular nibs 380 to prevent or reduce a too-fast ejection of the cardiac support system 600 from the tip 558, sometimes referred to as a watermelon seed effect. Peaks of a distal-most circumferential ring of the expandable housing 640 expand outwardly compared to valleys of the circumferential ring. The impeller 608 of the cardiac support system 600 also begins to self-expand as it emerges from the tip 558. In some arrangements the impeller 608 can expand faster than the expandable housing 640 such that the impeller 608 can contact and apply an expansion force to the expandable housing 640. In other arrangements the expandable housing 640 expands faster such that the expansion of the impeller 608 does not significantly affect the expansion of the expandable housing 640.

FIG. 16D shows a later stage of a method of deploying the cardiac support system 600 when the expandable housing 640 and the impeller 608 have been fully expanded. In this stage of delivery illustrated in this figure a motor assembly 624 of the cardiac support system 600 is still located inside the sheath 554, e.g., proximal of the tip 558 while the expandable housing 640 is disposed distal of the motor assembly 624. A portion of the struts making up the expandable housing 640 can extend across the tip 558 between the expanded portion of the expandable housing 640 and the motor assembly 624. In this position the circular nibs 380 and the peaks of the distal-most circumferential ring can be engaged with tissue of the wall of the vessel in which the cardiac support system 600 is placed.

FIG. 16E shows a later stage of the method of positioning the cardiac support system 600 in a blood vessel. In this stage further relative motion between the cardiac support system 600 and the delivery system moves the motor assembly 624 out of the sheath 554 distal to the tip 558. In this stage the power lead 626 extends from the motor assembly 624 across the tip 558 of the sheath 554 and to the proximal end of the delivery system. The power lead 626 can be connected to a controller outside of the patient. In a chronic use the power lead 626 can be connected to a source of power or current disposed beneath the skin, e.g., similar to the power supply device 276 as part of the power assembly 268.

FIG. 16E shows that the cardiac support system 600 can have unique features. For example, the impeller 608 can include a blade frame 620 disposed between a proximal ring 612 and a distal ring 616. The impeller 608 can include two blade frames 620 disposed opposite to each other, e.g., 180 degrees apart. The blade frames 620 can be provided on opposite sides of the torque shaft assembly 632 without any blade frames being disposed between the two opposed blade frames 620. By combining providing only two blade frames 620 (and corresponding tensile structure to move or be moved by the blood) and by providing a low angle between the distal and proximal ends of the blade the impeller 608 can be minimally disruptive to blood flow in a vessel. Also, by making the distal end of the expandable housing 640 self-supporting blood flow into the volume housing the impeller 608 can be substantially unobstructed. This combination of features makes the cardiac support system 600 less obstructive to blood passing by these components such that the cardiac support system 600 will not enhance a burden on the heart when not operational.

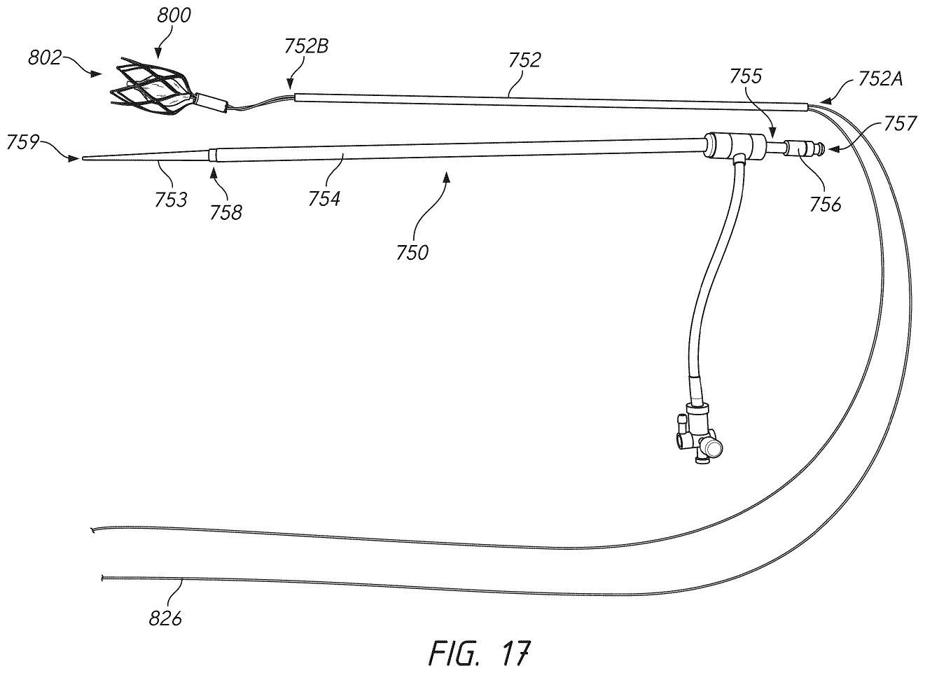

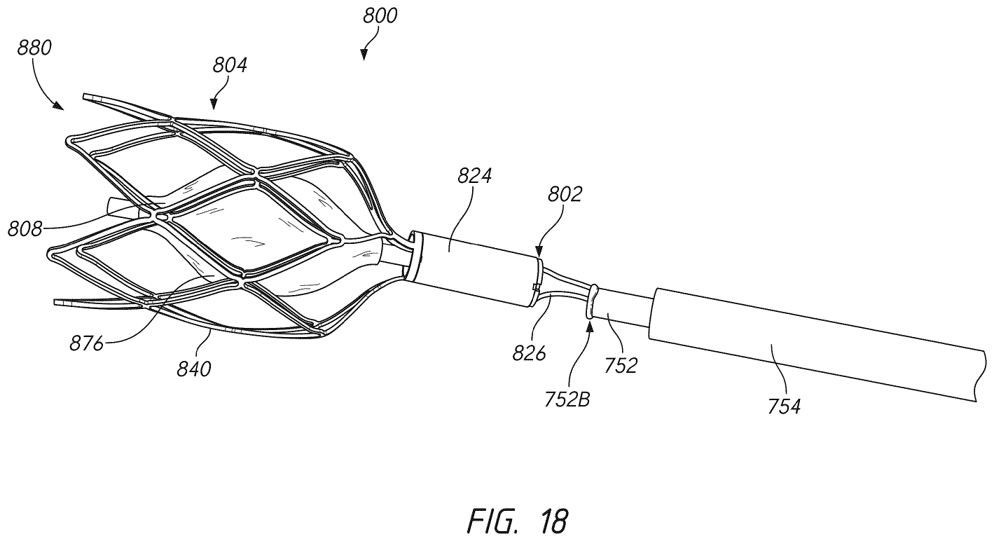

FIGS. 17 and 18 illustrate another cardiac support system 800 as well as a delivery system 750 that can deploy the system 800. Common features between the cardiac support system 600 and the cardiac support system 800 will not be described again but are incorporated here in their entirety, and common features between the delivery system 550 and the delivery system 750 will not be described again but are incorporated here in their entirety. In this embodiment, as shown in FIG. 17, the delivery system 750 can comprise a sheath 754 and a dilator 756. The sheath 754 may extend between a proximal end 755 and a tip 758. The dilator 756 may extend between a proximal end 757 and a dilator tip 759. The dilator tip 759 may be inserted into the sheath 754 at the proximal end 755 of the sheath 754. When the dilator 756 is inserted into the sheath 754, a distal portion 753 of the dilator 756 can be inserted through the sheath 754 so the distal portion 753 can extend distally from the tip 758 of the sheath 754. The distal portion 753 of the dilator 756 can comprise a tapered configuration. The tapered configuration of the distal portion 753 can stretch a vascular access point (e.g., in a femoral artery, a subclavian artery, an iliac artery, a femoral vein, an iliac vein, a radial artery, a radial vein, etc.) and/or the vasculature so the sheath 754 may be inserted through the vascular access point and/or the vasculature. The tapered configuration of the distal portion 753 can reduce, prevent, or inhibit the delivery system 750 from causing damage to the vascular access point and/or the vasculature.

The sheath 754 and the dilator 756 can be inserted into the vascular access point and moved through the vasculature until the tip 758 reaches an appropriate position, e.g., upstream of a renal artery, downstream of a renal vein or in another position in the venous and/or arterial system useful for enhancing diuresis and/or supporting heart function in another way. After the tip 758 of the sheath 754 has reached the appropriate position, the dilator 756 can be removed from the sheath 754. The dilator 756 can be removed from the proximal end 755 of the sheath 754. The dilator 756 can be removed from the sheath 754 by applying a load to (e.g., pulling) the proximal end 757 of the dilator 756. The distal portion 753 of the dilator 756 can translate or retract into the sheath 754 when the load is applied to the dilator 756. The sheath 754 can be held in a stationary position (e.g., by applying a load to the sheath 754) when the dilator 756 is removed from the sheath 754 so the tip 758 of the sheath 754 remains in the appropriate position.

After the dilator 756 is removed from the sheath 754, the cardiac support system 800 can be deployed. The cardiac support system 800 can be inserted into the sheath 754 at the proximal end 755 of the sheath 754. The cardiac support system 800 can be held in the crimped state when the cardiac support system 800 is inserted into the sheath 754. The cardiac support system 800 can be inserted into the sheath 754 so the expandable housing 840 of the fluid pump 804 is positioned distal to the motor assembly 824 of the fluid pump 804 when the cardiac support system 800 is deployed.

The cardiac support system 800 can moved through the sheath 754 by applying a load to a tool interface (e.g., the circumferentially narrowed section 149, discussed above, and/or a proximal end 802 of the motor assembly 824 of the cardiac support system 800). The cardiac support system 800 can be moved through the sheath 754 until the cardiac support system 800 reaches the tip 758 of the sheath 754. After the cardiac support system 800 reaches the tip 758 of the sheath 754, the cardiac support system 800 can be deployed similar to the cardiac support system 600, as described above with reference to FIGS. 16A-16E.

The delivery system 750 can comprise a deployment tool 752 (e.g., a pusher) that extends between a proximal end 752A and a distal end 752B. The deployment tool 752 can be configured as a tube-shaped elongate body that is sufficiently pushable to be moved through the sheath 754 by applying a load to the proximal end 752A. The deployment tool 752 can comprise a flexible and/or a stiff material, for example, radiopaque polyethylene, polytetrafluoroethylene (Teflon), stainless steel, or the like.

The deployment tool 752 can be positioned so the distal end 752B of the deployment tool 752 can apply a load to the tool interface of the cardiac support system 800. The power lead 826 of the cardiac support system 800 can be inserted into the deployment tool 752 and the deployment tool 752 can be moved along a length of the power lead 826 until the distal end 752B is positioned at or near the tool interface of the cardiac support system 800 so the power lead 826 can extend through the proximal end 752A of the deployment tool 752.

The deployment tool 752 can be inserted into the proximal end 755 of the sheath 754 after the cardiac support system 800 so the deployment tool 752 is positioned proximal to the expandable housing 840 and the motor assembly 824. A load can be applied to the deployment tool 752 at or near the proximal end 752A of the deployment tool 752 to move the deployment tool 752 and the cardiac support system 800 through the sheath 754 (i.e., from the proximal end 755 to the tip 758).

After the cardiac support system 800 reaches the tip 758 of the sheath 754, the deployment tool 752 can apply a load to the tool interface of the cardiac support system 800 to deploy the cardiac support system 800 similar to the cardiac support system 600, as described above with reference to FIGS. 16A-16E.

After the cardiac support system 800 is deployed, the deployment tool 752 can be removed from the sheath 754. The deployment tool 752 can be removed from the proximal end of the sheath 754. The deployment tool 752 can be removed from the sheath 754 by applying a load to (e.g., pulling) the proximal end 752A of the deployment tool 752. The sheath 754 can be held in a stationary position (e.g., by applying a load to the sheath 754) when the deployment tool 752 is removed from the sheath 754. After the deployment tool 752 is removed from the sheath 754, the sheath 754 may be removed from the vasculature by applying a load to (e.g., pulling) the proximal end 755 of the sheath 754. In some configurations, the sheath 754 and the deployment tool 752 can be removed from the vasculature simultaneously (e.g., with the deployment tool 752 positioned in the sheath).

FIG. 18 shows the cardiac support system 800. The cardiac support system 800 can comprise an impeller 808. The impeller 808 can comprise an impeller blade frame (not shown) and a tensile structure 876 coupled to the impeller blade frame. The tensile structure 876 can wrap around the outside of the impeller blade frame. When the cardiac support system 800 and the impeller 808 expand from the crimped state to the expanded state 880, the impeller blade frame can apply a radially outward force to the tensile structure 876 to stretch the tensile structure 876 and form each blade of the impeller 808.

The power lead 826 can be coupled or connected to the motor assembly 824 at the proximal end 802 of the motor assembly 824. The proximal end 802 of the motor assembly 824 can have potting. The potting can isolate the power lead 826 from blood pass by the power lead 826. The potting can provide strain relief to reduce or eliminate stresses applied to the power lead 826 at the connection between the power lead 826 and the motor assembly 824. The potting can prevent or inhibit the power lead 826 from being decoupled or disconnected from the motor assembly 824. The potting can comprise a polymer, epoxy, adhesive, or the like.

### Additional Embodiments

Embodiments of the present invention may be in accordance with any of the following clauses:

Clause 1. A method for increasing cardiac output of a heart of a patient and/or diuresis, comprising placing a fluid pump within a blood vessel of the patient, powering the fluid pump to rotate an impeller of the fluid pump in a first direction, and switching the fluid pump to an unpowered state wherein the impeller is rotated by blood flowing through the fluid pump and/or is non-obstructive to at least some blood flowing between impeller blades of the impeller.

Clause 2. The method of Clause 1, wherein an obstructive effect of the impeller of the fluid pump to a total volume of a blood stream flowing through the fluid pump when disposed in the blood vessel is reduced compared to the obstructive effect of the impeller if held stationary in the blood stream.

Clause 3. The method of Clause 2, wherein the fluid pump is configured such that blood in the blood vessel rotates the impeller of the fluid pump in a second direction opposite the first direction in the unpowered state.

Clause 4. The method of Clause 3, wherein the fluid pump is configured such that the fluid pump generates current to another device upon rotation of the impeller in the second direction in the unpowered state.

Clause 5. The method of Clause 1, wherein placing the fluid pump further comprises placing the fluid pump in an aorta of the patient and powering the fluid pump to rotate the impeller in the first direction improves cardiac output.

Clause 6. The method of Clause 5, wherein powering the fluid pump to rotate the impeller in the first direction transports blood away from an area adjacent to an aortic valve of the heart of the patient.

Clause 7. The method of Clause 1, wherein placing the fluid pump comprises placing the fluid pump in an aorta adjacent to or within a renal artery of the patient and powering the fluid pump to rotate the impeller in the first direction increases blood flow through the renal artery into kidneys of the patient to increase diuresis.

Clause 8. The method of Clause 1, wherein placing the fluid pump comprises placing the fluid pump in a portion of a vena cava and powering the fluid pump to rotate the impeller improves cardiac output.

Clause 9. The method of Clause 8, wherein powering the fluid pump to rotate the impeller of the fluid pump in the first direction comprises generating outflow to resist native blood flow in the portion of the vena cava.

Clause 10. The method of Clause 8, wherein placing the fluid pump comprises placing the fluid pump adjacent to or in a renal vein of the patient to enhance flow through a kidney of the patient by decreasing renal venous pressure.

Clause 11. The method of Clause 1, wherein placing the fluid pump comprises placing the fluid pump adjacent to or within a thoracic duct of the patient to modulate flow from the thoracic duct to the blood vessel.

Clause 12. The method of Clause 11, wherein powering the fluid pump to rotate the impeller increases flow of fluid out of the thoracic duct to increase flow in the thoracic duct to reduce interstitial fluid pressure.

Clause 13. The method of Clause 11, wherein powering the fluid pump to rotate the impeller impedes flow of fluid out of the thoracic duct to reduce fluid volume contribution from the thoracic duct to the blood vessel.

Clause 14. The method of Clause 1, further comprising supplying current from an implantable device to power the fluid pump.

Clause 15. The method of Clause 14, wherein the implantable device comprises an inductive charging receiver coil assembly comprising an inductive coil and, optionally, a battery and further comprising supplying current comprises supplying current generated in the inductive coil and, optionally, stored in the battery of the inductive charging receiver coil assembly.

Clause 16. The method of Clause 14, further comprising applying the inductive coil to a skin surface of the patient.

Clause 17. The method of Clause 16, wherein applying the inductive coil comprises applying a dermal sticker to a skin surface of the patient.

Clause 18. The method of Clause 16, wherein applying the inductive coil comprises tattooing a skin surface to form the inductive coil.

Clause 19. The method of Clause 1, wherein a gap is provided between a radial direction of the first end of one blade and a radial direction of a second end an adjacent blade.

Clause 20. The method of Clause 19, wherein an angle between about 75 degrees and about 120 degrees is provided between the radial direction of the first end of one blade and the radial direction of the second end the adjacent blade.

Clause 21. The method of Clause 1, wherein placing the fluid pump comprises the steps of: inserting a sheath into the blood vessel, wherein the sheath extends between a proximal end and a tip; inserting the fluid pump into the sheath at the proximal end; applying, via a pusher, a load to the fluid pump to move the fluid pump through the sheath to the tip of the sheath; deploying the fluid pump at the tip of the sheath; and removing the sheath from the blood vessel.

Clause 22. The method of Clause 1, wherein when the impeller rotates in the first direction, the fluid pump is configured to generate fluid flow in the blood vessel up to 5 L/min.

Clause 23. The method of Clause 1, wherein when the impeller rotates in the first direction, the fluid pump is configured to reduce pressure in the blood vessel upstream from the fluid pump when compared to a pressure in the blood vessel when no fluid pump is place in the blood vessel.

Clause 24. The method of Clause 1, wherein when the impeller rotates in the first direction, the fluid pump is configured to increase pressure in the blood vessel upstream from the fluid pump when compared to a pressure in the blood vessel when no fluid pump is place in the blood vessel.

Clause 25. The method of Clause 1, wherein placing the fluid pump further comprises placing the fluid pump in a vena cava of the patient upstream from an outflow of a renal vein from kidneys of the patient and powering the fluid pump to rotate the impeller in the first direction resists flow from upstream of the renal vein.

Clause 26. The method of Clause 25, further comprising powering the fluid pump to rotate the impeller of the fluid pump in the first direction to increase a proportion of flow in the vena cava from the kidneys of the patient.

Clause 27. The method of Clause 25, further comprising powering the fluid pump to rotate the impeller of the fluid pump in the first direction to decrease a proportion of flow from legs of the patient.

Clause 28. A system for chronic support of heart function, comprising: a motor sized for insertion into a blood vessel, the motor comprising windings to generate magnetic fields when energized; a torque shaft assembly comprising a torque shaft and a rotor, the rotor configured to be rotated in response to the magnetic fields; an expandable housing having a first end coupled with the motor, a second end opposite the first end, and a stent body disposed between the first end and the second end; and an expandable propeller disposed in the expandable housing, the expandable propeller comprising at least one propeller blade frame having a first end fixed to the torque shaft and a second end opposite the first end, the second end slideable along the torque shaft, the expandable propeller further comprising a tensile structure disposed along the at least one propeller blade frame and extending radially inwardly therefrom in an expanded state of the expandable propeller, wherein an angle of 45 degrees or less is provided between a radial direction of the first end and a radial direction of the second end; wherein when the windings of the motor are not generating magnetic fields, the expandable propeller is configured to freely rotate in response to blood flow in the blood vessel.

Clause 29. The system of Clause 28, wherein an angle of 30 degrees or less is provided between the radial direction of the first end and the radial direction of the second end.

Clause 30. The system of Clause 28, wherein the system in inserted into the blood vessel with a deployment system comprising a sheath, and a pusher, and wherein the system is inserted into a proximal end of the sheath when the sheath is inserted in the blood vessel, and a load is applied to the system, via the pusher, to move the system from the proximal end of the sheath to a tip of the sheath, and wherein the system is deployed in the blood vessel at the tip of the sheath.

Clause 31. A system for enhancing cardiac output and/or diuresis through enhanced cardiorenal flow, comprising: a battery; and a pump-generator unit, comprising: a housing comprising at least one wire coil assembly configured to convey current in response to a magnetic field and/or to generate a magnetic field in response to current conveyed therein; an expandable stent having a first end coupled with the housing, a second end opposite the first end, and a stent body disposed between the first end and the second end; a shaft assembly comprising a shaft at least partially disposed in the expandable stent and a rotor rotatably coupled with the housing; and at least one blade frame having a first end fixed to the shaft and a second end opposite the first end, the second end slideable along the shaft, a tensile structure disposed along the at least one blade frame and extending radially inwardly therefrom in an expanded state of the at least one blade frame and the tensile structure; wherein in one operating state, blood flow onto the tensile structure applies a load to the tensile structure resulting in a torque applied to the shaft and the rotor, the rotor rotating in response to the torque causing a magnet in the rotor to generate a magnetic field to create a current in the at least one wire coil assembly, the current directed to the battery to charge the battery.

Clause 32. The system of Clause 31, further comprising an intravascular blood pump configured to be coupled with the pump-generator unit, the intravascular blood pump comprising a motor electrically connected to the battery and configured to operate using current from the battery.

Clause 33. The system of Clause 32, wherein the intravascular blood pump is configured to be positioned downstream of renal veins of a patient and the pump-generator unit is configured to be positioned upstream of the renal veins of the patient.

Clause 34. A system for chronic support of heart function, comprising: a pump assembly comprising an expandable stent, a motor coupled with a proximal end of the expandable stent, and a propeller disposed on a torque shaft disposed in the expandable stent; a power lead coupled with the motor at a distal end and having a proximal end disposed opposite the distal end, the power lead having a length between the proximal end and the distal end sufficient to enable the pump assembly to be disposed in a blood vessel of a patient when the proximal end is disposed outside of a peripheral vessel in fluid communication with the blood vessel; and a coil assembly comprising a support member enclosing an inductive coil, the inductive coil configured to couple with the proximal end of the power lead, the support member configured to be implanted minimally subcutaneously.

Clause 35. The system of Clause 34, wherein the coil assembly is a secondary coil assembly and further comprising a primary coil assembly configured to be coupled with the patient over the secondary coil assembly and to transfer power transdermally to the secondary coil assembly to provide current to the motor.

Clause 36. The system of Clause 35, wherein the primary coil assembly is configured to transfer power to the secondary coil assembly across a distance of about 5 mm and about 50 mm.

Clause 37. The system of Clause 35, wherein the primary coil assembly is configured to transfer power to the secondary coil assembly across a distance of about 10 mm and about 40 mm.

Clause 38. The system of Clause 35, wherein the primary coil assembly is configured to transfer power to the secondary coil assembly across a distance of about 20 mm and about 30 mm.

Clause 39. The system of Clause 35, wherein the primary coil assembly is configured to transfer power to the secondary coil assembly across a distance of about 24 mm.

Clause 40. The system of Clause 35, further comprising a battery configured to provide current to the motor and to be charged by current generated by inductive coupling of the secondary coil assembly with the primary coil assembly.

Clause 41. The system of Clause 40, further comprising a motor housing disposed around the motor and the battery.

Clause 42. The system of Clause 40, wherein the battery is disposed adjacent to the secondary coil assembly and to the proximal end of the power lead.

Clause 43. The system of Clause 34, wherein the propeller comprises an expandable structure configured to be actuated to a compressed state for introduction in the patient and to an expanded state within the patient, the expanded state configured to cause the propeller to pump blood when rotated.

Clause 44. The system of Clause 43, wherein the expandable structure comprises a frame extending from a support fixed to the torque shaft along the torque shaft forming a radially outer periphery of the propeller and a covering extending from the frame to a central area of the propeller.

Clause 45. The system of Clause 34, wherein the propeller is fixed to the torque shaft one end and slideable over the torque shaft on an end opposite the end fixed to the torque shaft.

Clause 46. The system of Clause 34, wherein the propeller comprises a helical element configured to pump blood.

Clause 47. The system of Clause 34, wherein the expandable stent comprises a plurality of expandable circumferential rings.

Clause 48. The system of Clause 47, wherein at least one expandable circumferential ring of the plurality of expandable circumferential rings comprises an undulating structure comprising a plurality of proximal apices and a plurality of distal apices wherein adjacent apices are spaced by a first amount in a compressed state and a second amount in an expanded state, the second amount greater than the first amount.

Clause 49. The system of Clause 47, wherein at least one expandable circumferential ring of the plurality of expandable circumferential rings comprise a plurality of apices configured to locally deflect out of cylinder when the expandable stent is in an expanded state.

Clause 50. The system of Clause 49, wherein the plurality of apices configured to locally deflect are distal apices, further comprising a plurality of proximal apices adjacent to distal apices being connected to proximal-distal support struts of the expandable stent.

Clause 51. The system of Clause 34, wherein the expandable stent comprises a plurality of closed cells disposed between proximal and distal ends of the expandable stent.

Clause 52. The system of Clause 34, wherein the propeller comprises a frame disposed around a periphery, the periphery comprising a proximal strut portion, an axial strut portion, and a distal strut portion, an angle between the proximal strut portion and the distal strut portion as seen from the distal end is less than 30 degrees.

Clause 53. The system of Clause 34, wherein the support member of the coil assembly is configured to be implanted adjacent to or over the peripheral vessel.

Clause 54. The system of Clause 53, wherein the support member of the coil assembly is configured to be implanted adjacent to or over an iliac artery, a subclavian artery, or a femoral artery.

Clause 55. The system of Clause 34, wherein the support member of the coil assembly comprises an adhesive backed conformal member configured to adhere to a patients skin.

Clause 56. A system for chronic support of heart function, comprising: a motor sized for insertion into a patient; a shaft assembly rotatably coupled with the motor, the shaft assembly including a torque shaft; an expandable housing having a first end disposed about at least a portion of the shaft assembly, a second end opposite the first end, the second end being circumferentially self-supporting without axial or radial struts and thereby open to blood flow into or out of the expandable housing, and a stent body disposed between the first end and the second end; and an expandable propeller disposed in the expandable housing, the expandable propeller comprising at least one propeller blade frame having a first end fixed to the torque shaft and a second end opposite the first end, the expandable propeller further comprising a tensile structure disposed along the at least one propeller blade frame and extending radially inwardly therefrom in an expanded state of the expandable propeller.

Clause 57. The system of Clause 56, wherein the second end of the at least one propeller blade is coupled with and slideable along the torque shaft.

Clause 58. The system of Clause 56, wherein the motor is coupled to the expandable housing and sized for insertion into a blood vessel of the patient.

Clause 59. The system of Clause 56, wherein the motor is configured to be implanted beneath a skin surface of the patient, the shaft assembly comprising a drive cable coupled with the motor at a first end and with the torque shaft at a second end opposite the first end.

Clause 60. The system of Clause 56, wherein an angle of 45 degrees or less is provided between a radial direction of the first end and a radial direction of the second end.

Clause 61. The system of Clause 50, wherein the angle and/or a length between the first end and the second end of the at least one propeller blade frame is modified based on an RPM of the propeller, a fluid flow rate generated by the expandable propeller, and/or a fluid pressure in a blood vessel.

Clause 62. The system of Clause 56, wherein the motor comprises windings configured to generate magnetic fields to cause rotation of the torque shaft in response to the magnetic fields, wherein when the windings of the motor are not generating magnetic fields, the expandable propeller is configured to freely rotate in response to blood flow in a blood vessel in which the expandable propeller is disposed.

Clause 63. The system of Clause 56, further comprising a charging system comprising a power supply device configured to be implanted in a patient and to generate current or store power in response to exposure to an energy source disposed outside of the patient.

Clause 64. The system of Clause 63, wherein the power supply device comprises a coil assembly configured to generate current by induction in response to magnetic fields generated by the energy source.

Clause 65. The system of Clause 63, wherein the power supply device comprises a piezoelectric actuator configured to generate current in response to sound waves generated by the energy source.

Clause 66. The system of Clause 63, wherein the power supply device comprises a piezoelectric member disposed around and/or coupled with the motor, the piezoelectric member generating current or motion in response to sound waves generated by the energy source.

Clause 67. The system of Clause 63, wherein the energy source comprises an infrared transmitter configured to direct light energy restricted to the infrared range of the electromagnetic spectrum and the power supply device comprises an implantable infrared receiver configured to detect the light energy and to convert the light energy into current.

Clause 68. The system of Clause 67, further comprising a target feature configured to be detected by the infrared transmitter, the target feature indicating a location of the implantable infrared receiver when the implantable infrared receiver is implanted.

Clause 69. The system of Clause 68, wherein the target feature comprises a pattern configured to be applied to a portion of skin of the patient above an implantation site of the implantable infrared receiver.

Clause 70. The system of Clause 63, wherein the energy source comprises a radiofrequency transmitter configured to generate radio waves in a wavelength of 20-300 GHz and the power supply device comprises a receiver configured to detect the radio waves in the wavelength of 20-300 GHz and to convert the radio waves into current.

Clause 71. A system for enhancing cardiac output and/or diuresis through enhanced cardiorenal flow, comprising: a power source; and a pump, comprising: a housing comprising at least one wire coil assembly configured to convey current in response to a magnetic field and/or generate a magnetic field in response to current conveyed therein; an expandable stent having a first end coupled with the housing, a second end opposite the first end, and a stent body disposed between the first end and the second end; a shaft assembly comprising a shaft at least partially disposed in the expandable stent and a rotor rotatably coupled with the housing; and an impeller coupled to the shaft assembly, the impeller comprising at least one blade having toroidal configuration; wherein the power source is configured to convey current to the at least one wire coil assembly so the wire coil assembly rotates the shaft assembly and the impeller.

Clause 72. The system of Clause 71, wherein the at least one blade comprises a first end fixed to the shaft assembly, and a second end opposite the first end, the at least one blade extending radially outward from the shaft at the first end, and the at least one blade curving back towards the shaft so the at least one blade is fixed to the shaft assembly at the second end, wherein the at least one blade forms a gap between the at least one blade and the shaft assembly between the first end and the second end.

Clause 73. The system of Clause 71, wherein the at least one blade is formed by cutting or shaping nitinol into the toroidal configuration.

### Other Variations and Terminology

Features, materials, characteristics, or groups described in conjunction with a particular aspect, embodiment, or example are to be understood to be applicable to any other aspect, embodiment or example described herein unless incompatible therewith. All of the features disclosed in this specification (including any accompanying claims, abstract and drawings), or all of the steps of any method or process so disclosed, may be combined in any combination, except combinations where at least some of such features or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

While certain embodiments have been described, these embodiments have been presented by way of example only and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some embodiments, the actual steps taken in the processes illustrated or disclosed may differ from those shown in the figures. Depending on the embodiment, certain of the steps described above may be removed, others may be added. For example, the actual steps or order of steps taken in the disclosed processes may differ from those shown in the figure. Depending on the embodiment, certain of the steps described above may be removed, others may be added. Furthermore, the features and attributes of the specific embodiments disclosed above may be combined in different ways to form additional embodiments, all of which fall within the scope of the present disclosure.

Although the present disclosure includes certain embodiments, examples and applications, it will be understood by those skilled in the art that the present disclosure extends beyond the specifically disclosed embodiments to other alternative embodiments or uses and obvious modifications and equivalents thereof, including embodiments which do not provide all of the features and advantages set forth herein. Accordingly, the scope of the present disclosure is not intended to be limited by the described embodiments and may be defined by claims as presented herein or as presented in the future.

Conditional language, such as “can,” “could,” “might,” or “may,” unless specifically stated otherwise, or otherwise understood within the context as used, is generally intended to convey that certain embodiments include, while other embodiments do not include certain features, elements, or steps. Thus, such conditional language is not generally intended to imply that features, elements, or steps are in any way required for one or more embodiments or that one or more embodiments necessarily include logic for deciding, with or without user input or prompting, whether these features, elements, or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Likewise, the term “and/or” in reference to a list of two or more items, covers all of the following interpretations of the word: any one of the items in the list, all of the items in the list, and any combination of the items in the list. Further, the term “each,” as used herein, in addition to having its ordinary meaning, can mean any subset of a set of elements to which the term “each” is applied. Additionally, the words “herein,” “above,” “below,” and words of similar import, when used in this application, refer to this application as a whole and not to any particular portions of this application.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately,” “about,” “generally,” and “substantially” may refer to an amount that is within less than 10% of, within less than 5% of, within less than 1% of, within less than 0.1% of, and within less than 0.01 % of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

What is claimed is:

1. A method for increasing cardiac output of a heart of a patient and/or diuresis, comprising placing a fluid pump within a blood vessel of the patient, powering the fluid pump to rotate an impeller of the fluid pump in a first direction, and switching the fluid pump to an unpowered state wherein the impeller non-obstructive to at least some blood flowing between impeller blades of the impeller.

2. The method of claim 1, wherein an obstructive effect of the impeller of the fluid pump to a total volume of a blood stream flowing through the fluid pump when disposed in the blood vessel is reduced compared to the obstructive effect of the impeller if held stationary in the blood stream.

3. The method of claim 2, wherein the fluid pump is configured such that blood in the blood vessel rotates the impeller of the fluid pump in a second direction opposite the first direction in the unpowered state.

4. The method of claim 3, wherein the fluid pump is configured such that the fluid pump generates current to another device upon rotation of the impeller in the second direction in the unpowered state.

5. The method of claim 1, wherein placing the fluid pump further comprises placing the fluid pump in an aorta of the patient and powering the fluid pump to rotate the impeller in the first direction improves cardiac output.

6. The method of claim 5, wherein powering the fluid pump to rotate the impeller in the first direction transports blood away from an area adjacent to an aortic valve of the heart of the patient.

7. The method of claim 1, wherein placing the fluid pump comprises placing the fluid pump in an aorta adjacent to or within a renal artery of the patient and powering the fluid pump to rotate the impeller in the first direction increases blood flow through the renal artery into kidneys of the patient to increase diuresis.

8. The method of claim 1, wherein placing the fluid pump comprises placing the fluid pump in a portion of a vena cava and powering the fluid pump to rotate the impeller improves cardiac output.

9. The method of claim 8, wherein powering the fluid pump to rotate the impeller of the fluid pump in the first direction comprises generating outflow to resist native blood flow in the portion of the vena cava.

10. The method of claim 8, wherein placing the fluid pump comprises placing the fluid pump adjacent to or in a renal vein of the patient to enhance flow through a kidney of the patient by decreasing renal venous pressure.

11. The method of claim 1, wherein placing the fluid pump comprises placing the fluid pump adjacent to or within a thoracic duct of the patient to modulate flow from the thoracic duct to the blood vessel.

12. The method of claim 11, wherein powering the fluid pump to rotate the impeller increases flow of fluid out of the thoracic duct to increase flow in the thoracic duct to reduce interstitial fluid pressure.

13. The method of claim 11, wherein powering the fluid pump to rotate the impeller impedes flow of fluid out of the thoracic duct to reduce fluid volume contribution from the thoracic duct to the blood vessel.

14. The method of claim 1, further comprising supplying current from an implantable device to power the fluid pump.

15. The method of claim 14, wherein the implantable device comprises an inductive charging receiver coil assembly comprising an inductive coil and, optionally, a battery and further comprising supplying current comprises supplying current generated in the inductive coil and, optionally, stored in the battery of the inductive charging receiver coil assembly.

16. The method of claim 14, further comprising applying the inductive coil to a skin surface of the patient.

17. The method of claim 16, wherein applying the inductive coil comprises applying a dermal sticker to a skin surface of the patient.

18. The method of claim 16, wherein applying the inductive coil comprises tattooing a skin surface to form the inductive coil.

19. The method of claim 1, wherein a gap is provided between a radial direction of the first end of one blade and a radial direction of a second end an adjacent blade.

20. The method of claim 19, wherein an angle between about 75 degrees and about 120 degrees is provided between the radial direction of the first end of one blade and the radial direction of the second end the adjacent blade.

21. The method of claim 1, wherein placing the fluid pump comprises the steps of:
inserting a sheath into the blood vessel, wherein the sheath extends between a proximal end and a tip;
inserting the fluid pump into the sheath at the proximal end;
applying, via a pusher, a load to the fluid pump to move the fluid pump through the sheath to the tip of the sheath;
deploying the fluid pump at the tip of the sheath; and
removing the sheath from the blood vessel.

22. The method of claim 1, wherein when the impeller rotates in the first direction, the fluid pump is configured to generate fluid flow in the blood vessel up to 5 L/min.

23. The method of claim 1, wherein when the impeller rotates in the first direction, the fluid pump is configured to reduce pressure in the blood vessel upstream from the fluid pump when compared to a pressure in the blood vessel when no fluid pump is place in the blood vessel.

24. The method of claim 1, wherein when the impeller rotates in the first direction, the fluid pump is configured to increase pressure in the blood vessel upstream from the fluid pump when compared to a pressure in the blood vessel when no fluid pump is place in the blood vessel.

25. The method of claim 1, wherein placing the fluid pump further comprises placing the fluid pump in a vena cava of the patient upstream from an outflow of a renal vein from kidneys of the patient and powering the fluid pump to rotate the impeller in the first direction resists flow from upstream of the renal vein.

26. The method of claim 25, further comprising powering the fluid pump to rotate the impeller of the fluid pump in the first direction to increase a proportion of flow in the vena cava from the kidneys of the patient.

27. The method of claim 25, further comprising powering the fluid pump to rotate the impeller of the fluid pump in the first direction to decrease a proportion of flow from legs of the patient.

28. A system for chronic support of heart function, comprising:
a motor sized for insertion into a blood vessel, the motor comprising windings to generate magnetic fields when energized;
a torque shaft assembly comprising a torque shaft and a rotor, the rotor configured to be rotated in response to the magnetic fields;
an expandable housing having a first end coupled with the motor, a second end opposite the first end, and a stent body disposed between the first end and the second end; and
an expandable propeller disposed in the expandable housing, the expandable propeller comprising at least one propeller blade frame having a first end fixed to the torque shaft and a second end opposite the first end, the second end slideable along the torque shaft, the expandable propeller further comprising a tensile structure disposed along the at least one propeller blade frame and extending radially inwardly therefrom in an expanded state of the expandable propeller, wherein an angle of 45 degrees or less is provided between a radial direction of the first end and a radial direction of the second end.

29. The system of claim 28, wherein an angle of 30 degrees or less is provided between the radial direction of the first end and the radial direction of the second end.

30. The system of claim 28, wherein the system in inserted into the blood vessel with a deployment system comprising a sheath, and a pusher, and wherein the system is inserted into a proximal end of the sheath when the sheath is inserted in the blood vessel, and a load is applied to the system, via the pusher, to move the system from the proximal end of the sheath to a tip of the sheath, and wherein the system is deployed in the blood vessel at the tip of the sheath.

31. A system for enhancing cardiac output and/or diuresis through enhanced cardiorenal flow, comprising:
a battery; and
a pump-generator unit, comprising:
a housing comprising at least one wire coil assembly configured to convey current in response to a magnetic field and/or to generate a magnetic field in response to current conveyed therein;
an expandable stent having a first end coupled with the housing, a second end opposite the first end, and a stent body disposed between the first end and the second end;
a shaft assembly comprising a shaft at least partially disposed in the expandable stent and a rotor rotatably coupled with the housing; and
at least one blade frame having a first end fixed to the shaft and a second end opposite the first end, the second end slideable along the shaft, a tensile structure disposed along the at least one blade frame and extending radially inwardly therefrom in an expanded state of the at least one blade frame and the tensile structure;
wherein in one operating state, blood flow onto the tensile structure applies a load to the tensile structure resulting in a torque applied to the shaft and the rotor, the rotor rotating in response to the torque causing a magnet in the rotor to generate a magnetic field to create a current in the at least one wire coil assembly, the current directed to the battery to charge the battery.

32. The system of claim 31, further comprising an intravascular blood pump configured to be coupled with the pump-generator unit, the intravascular blood pump comprising a motor electrically connected to the battery and configured to operate using current from the battery.

33. The system of claim 32, wherein the intravascular blood pump is configured to be positioned downstream of renal veins of a patient and the pump-generator unit is configured to be positioned upstream of the renal veins of the patient.

34. A system for chronic support of heart function, comprising:
a pump assembly comprising an expandable stent, a motor coupled with a proximal end of the expandable stent, and a propeller disposed on a torque shaft disposed in the expandable stent;
a power lead coupled with the motor at a distal end and having a proximal end disposed opposite the distal end, the power lead having a length between the proximal end and the distal end sufficient to enable the pump assembly to be disposed in a blood vessel of a patient when the proximal end is disposed outside of a peripheral vessel in fluid communication with the blood vessel; and
a coil assembly comprising a support member enclosing an inductive coil, the inductive coil configured to couple with the proximal end of the power lead, the support member configured to be implanted minimally subcutaneously.

35. The system of claim 34, wherein the coil assembly is a secondary coil assembly and further comprising a primary coil assembly configured to be coupled with the patient over the secondary coil assembly and to transfer power transdermally to the secondary coil assembly to provide current to the motor.

36. The system of claim 35, wherein the primary coil assembly is configured to transfer power to the secondary coil assembly across a distance of about 5 mm and about 50 mm.

37. The system of claim 35, wherein the primary coil assembly is configured to transfer power to the secondary coil assembly across a distance of about 10 mm and about 40 mm.

38. The system of claim 35, wherein the primary coil assembly is configured to transfer power to the secondary coil assembly across a distance of about 20 mm and about 30 mm.

39. The system of claim 35, wherein the primary coil assembly is configured to transfer power to the secondary coil assembly across a distance of about 24 mm.

40. The system of claim 35, further comprising a battery configured to provide current to the motor and to be charged by current generated by inductive coupling of the secondary coil assembly with the primary coil assembly.

41. The system of claim 40, further comprising a motor housing disposed around the motor and the battery.

42. The system of claim 40, wherein the battery is disposed adjacent to the secondary coil assembly and to the proximal end of the power lead.

43. The system of claim 34, wherein the propeller comprises an expandable structure configured to be actuated to a compressed state for introduction in the patient and to an expanded state within the patient, the expanded state configured to cause the propeller to pump blood when rotated.

44. The system of claim 43, wherein the expandable structure comprises a frame extending from a support fixed to the torque shaft along the torque shaft forming a radially outer periphery of the propeller and a covering extending from the frame to a central area of the propeller.

45. The system of claim 34, wherein the propeller is fixed to the torque shaft one end and slideable over the torque shaft on an end opposite the end fixed to the torque shaft.

46. The system of claim 34, wherein the propeller comprises a helical element configured to pump blood.

47. The system of claim 34, wherein the expandable stent comprises a plurality of expandable circumferential rings.

48. The system of claim 47, wherein at least one expandable circumferential ring of the plurality of expandable circumferential rings comprises an undulating structure comprising a plurality of proximal apices and a plurality of distal apices wherein adjacent apices are spaced by a first amount in a compressed state and a second amount in an expanded state, the second amount greater than the first amount.

49. The system of claim 47, wherein at least one expandable circumferential ring of the plurality of expandable circumferential rings comprise a plurality of apices configured to locally deflect out of cylinder when the expandable stent is in an expanded state.

50. The system of claim 49, wherein the plurality of apices configured to locally deflect are distal apices, further comprising a plurality of proximal apices adjacent to distal apices being connected to proximal-distal support struts of the expandable stent.

51. The system of claim 34, wherein the expandable stent comprises a plurality of closed cells disposed between proximal and distal ends of the expandable stent.

52. The system of claim 34, wherein the propeller comprises a frame disposed around a periphery, the periphery comprising a proximal strut portion, an axial strut portion, and a distal strut portion, an angle between the proximal strut portion and the distal strut portion as seen from the distal end is less than 30 degrees.

53. The system of claim 34, wherein the support member of the coil assembly is configured to be implanted adjacent to or over the peripheral vessel.

54. The system of claim 53, wherein the support member of the coil assembly is configured to be implanted adjacent to or over an iliac artery, a subclavian artery, or a femoral artery.

55. The system of claim 34, wherein the support member of the coil assembly comprises an adhesive backed conformal member configured to adhere to a patients skin.

56. A system for chronic support of heart function, comprising:
a motor sized for insertion into a patient;
a shaft assembly rotatably coupled with the motor, the shaft assembly including a torque shaft;
an expandable housing having a first end disposed about at least a portion of the shaft assembly, a second end opposite the first end, the second end being circumferentially self-supporting without axial or radial struts and thereby open to blood flow into or out of the expandable housing, and a stent body disposed between the first end and the second end; and
an expandable propeller disposed in the expandable housing, the expandable propeller comprising at least one propeller blade frame having a first end fixed to the torque shaft and a second end opposite the first end, the expandable propeller further comprising a tensile structure disposed along the at least one propeller blade frame and extending radially inwardly therefrom in an expanded state of the expandable propeller.

57. The system of claim 56, wherein the second end of the at least one propeller blade is coupled with and slideable along the torque shaft.

58. The system of claim 56, wherein the motor is coupled to the expandable housing and sized for insertion into a blood vessel of the patient.

59. The system of claim 56, wherein the motor is configured to be implanted beneath a skin surface of the patient, the shaft assembly comprising a drive cable coupled with the motor at a first end and with the torque shaft at a second end opposite the first end.

60. The system of claim 56, wherein an angle of 45 degrees or less is provided between a radial direction of the first end and a radial direction of the second end.

61. The system of claim 50, wherein the angle and/or a length between the first end and the second end of the at least one propeller blade frame is modified based on an RPM of the propeller, a fluid flow rate generated by the expandable propeller, and/or a fluid pressure in a blood vessel.

62. The system of claim 56, wherein the motor comprises windings configured to generate magnetic fields to cause rotation of the torque shaft in response to the magnetic fields, wherein when the windings of the motor are not generating magnetic fields, the expandable propeller is configured to freely rotate in response to blood flow in a blood vessel in which the expandable propeller is disposed.

63. The system of claim 56, further comprising a charging system comprising a power supply device configured to be implanted in a patient and to generate current or store power in response to exposure to an energy source disposed outside of the patient.

64. The system of claim 63, wherein the power supply device comprises a coil assembly configured to generate current by induction in response to magnetic fields generated by the energy source.

65. The system of claim 63, wherein the power supply device comprises a piezoelectric actuator configured to generate current in response to sound waves generated by the energy source.

66. The system of claim 63, wherein the power supply device comprises a piezoelectric member disposed around and/or coupled with the motor, the piezoelectric member generating current or motion in response to sound waves generated by the energy source.

67. The system of claim 63, wherein the energy source comprises an infrared transmitter configured to direct light energy restricted to the infrared range of the electromagnetic spectrum and the power supply device comprises an implantable infrared receiver configured to detect the light energy and to convert the light energy into current.

68. The system of claim 67, further comprising a target feature configured to be detected by the infrared transmitter, the target feature indicating a location of the implantable infrared receiver when the implantable infrared receiver is implanted.

69. The system of claim 68, wherein the target feature comprises a pattern configured to be applied to a portion of skin of the patient above an implantation site of the implantable infrared receiver.

70. The system of claim 63, wherein the energy source comprises a radiofrequency transmitter configured to generate radio waves in a wavelength of 20-300 GHz and the power supply device comprises a receiver configured to detect the radio waves in the wavelength of 20-300 GHz and to convert the radio waves into current.

71. A system for enhancing cardiac output and/or diuresis through enhanced cardiorenal flow, comprising:
a power source; and
a pump, comprising:
a housing comprising at least one wire coil assembly configured to convey current in response to a magnetic field and/or generate a magnetic field in response to current conveyed therein;
an expandable stent having a first end coupled with the housing, a second end opposite the first end, and a stent body disposed between the first end and the second end;
a shaft assembly comprising a shaft at least partially disposed in the expandable stent and a rotor rotatably coupled with the housing; and
an impeller coupled to the shaft assembly, the impeller comprising at least one blade having toroidal configuration;
wherein the power source is configured to convey current to the at least one wire coil assembly so the wire coil assembly rotates the shaft assembly and the impeller.

72. The system of claim 71, wherein the at least one blade comprises a first end fixed to the shaft assembly, and a second end opposite the first end, the at least one blade extending radially outward from the shaft at the first end, and the at least one blade curving back towards the shaft so the at least one blade is fixed to the shaft assembly at the second end, wherein the at least one blade forms a gap between the at least one blade and the shaft assembly between the first end and the second end.

73. The system of claim 71, wherein the at least one blade is formed by cutting or shaping nitinol into the toroidal configuration.
